(19) United States
(12) Patent Application Publication
(10) Pub. No.: US 2022/0389115 A1
(43) Pub. Date: Dec. 8, 2022

(54) Methods of Treating Cancer with an Anti-CD39 Antibody

(71) Applicant: Surface Oncology, Inc., Cambridge, MA (US)

(72) Inventors: Andrew Lake, Westwood, MA (US); Alison M. O'NEILL, Cambridge, MA (US); Jou-Ku Chung, Cambridge, MA (US); Robert Ross, Cambridge, MA (US)

(73) Assignee: Surface Oncology, Inc., Cambridge, MA (US)

(21) Appl. No.: 17/829,480

(22) Filed: Jun. 1, 2022

Related U.S. Application Data
(60) Provisional application No. 63/286,291, filed on Dec. 6, 2021.
(60) Provisional application No. 63/252,280, filed on Oct. 5, 2021.
(60) Provisional application No. 63/196,456, filed on Jun. 3, 2021.

(51) Int. Cl. C07K 16/28; A61P 35/00; A61K 39/395; A61K 45/06; A61K 31/7068; A61K 47/64

(52) U.S. Cl. CPC C07K 16/2896; A61P 35/00; A61K 39/3955; A61K 45/06; A61K 31/7068; A61K 47/643; A61K 2039/505

(57) ABSTRACT

The invention provides methods of treating cancer using an anti-CD39 antibody or fragment thereof as monotherapy or in combination with other therapies at particular dosages and frequencies.

### CROSS-REFERENCE TO RELATED APPLICATIONS

This application claims priority to U.S. Provisional Application No. 63/286,291, filed Dec. 6, 2021, and U.S. Provisional Application No. 63/252,280, filed Oct. 5, 2021, and U.S. Provisional Application No. 63/196,456, filed Jun. 3, 2021, all of which are incorporated by reference in their entireties.

### SEQUENCE LISTING

This application contains a Sequence Listing in computer readable form entitled “01219-0008-00PCT_ST25”, created May 24, 2022, having a size of 1080000 Bytes, which is incorporated by reference herein.

### FIELD OF THE INVENTION

Methods of treating cancer with an anti-CD39 antibody at particular dosages are provided as a monotherapy or in combination with additional therapies.

### BACKGROUND

The immune system acts through suppressive pathways to prevent cancerous cells from growing. Cancers use various mechanisms to subvert immune suppressive pathways in order to avoid recognition and elimination by immune cells, and to allow disease to progress.

Immunotherapies fight cancer by modifying the patient's immune system by either directly stimulating rejection-type processes or by blocking suppressive pathways.

Adenosine is an immunomodulatory metabolite within the tumor microenvironment (TME) that interferes with the immune system's anti-tumor responses. In some cancers, extracellular adenosine accumulates and subsequently inhibits the function of immune cells, including T cells, dendritic cells (DC), and natural killer (NK) cells, thereby contributing to anti-tumor immune suppression and supporting tumor growth.

The ectonucleotidase CD39 hydrolyzes extracellular adenosine triphosphate (ATP) and adenosine diphosphate (ADP) to generate adenosine monophosphate (AMP), which is then converted to adenosine by CD73. Extracellular adenosine binds to adenosine receptors on immune cells, thereby suppressing the immune system. Overexpression of CD39 is associated with poor prognosis in patients with certain types of cancer. Within the TME, the adenosine pathway refers to the extracellular conversion of ATP to adenosine and the signaling of adenosine through the A2A/A2B adenosine receptors on immune cells. Under normal conditions, CD39 works to maintain the balance of extracellular levels of immunosuppressive adenosine and immunostimulatory ATP. In healthy tissues, ATP is barely detectable in the extracellular environment due to rapid breakdown by CD39 and conversion to adenosine by CD73. Under conditions of cellular stress, including cancer, extracellular ATP levels rise significantly leading to high levels of adenosine, which acts to suppress recognition of the tumor by the immune system and the anti-tumor response. While maintained levels of ATP increase T cell proliferation, dendritic cell maturation and pro-inflammatory cytokine levels, the accumulation of adenosine leads to immunosuppression. CD39 inhibition decreases immunosuppressive adenosine while stimulating immune responses by potentiating ATP levels in the TME.

There continues to be an unmet need for the development of methods of treating cancer. Novel combinations with existing therapies and therapeutic regimens are also needed to more effectively combat various cancers.

Provided herein are methods for treating cancer comprising administering an anti-CD39 antibody at particular dosages, e.g., administered as a flat (or fixed) dose between 20 and 2000 mg at particular dosing intervals. Pharmaceutical formulations are also provided for use in the disclosed methods. The methods and pharmaceutical formulations disclosed herein provide the benefit of treating various types of cancer, including relapsed or refractory solid tumors, while being well-tolerated, either as a monotherapy or as a combination therapy.

### SUMMARY

Embodiment A1 is a method of treating cancer in a human subject in need thereof comprising administering a pharmaceutical composition comprising an anti-CD39 antibody or fragment thereof, wherein the antibody is administered at a dose of 1400 mg or 2000 mg.

Embodiment A2 is the method of embodiment 1, wherein the anti-CD39 antibody or fragment thereof comprises:
a. HCDR1 comprising the amino acid sequence of SEQ ID NO: 30001;
b. HCDR2 comprising the amino acid sequence of SEQ ID NO: 30002;
c. HCDR3 comprising the amino acid sequence of SEQ ID NO: 30003;
d. LCDR1 comprising the amino acid sequence of SEQ ID NO: 30004;
e. LCDR2 comprising the amino acid sequence of SEQ ID NO: 30005; and
f. LCDR3 comprising the amino acid sequence of SEQ ID NO: 30006.

Embodiment A3 is the method of embodiment 1, wherein the antibody or fragment thereof is administered intravenously.

Embodiment A4 is the method of embodiment 1 or embodiment 2, wherein the antibody or fragment thereof is administered once every 1, 2, 3, 4, 5 or 6 weeks.

Embodiment A5 is the method of any one of embodiments 1-3, wherein the antibody or fragment thereof is administered once every 2 weeks.

Embodiment A6 is the method of any one of the preceding embodiments, wherein the antibody or fragment thereof comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 30012 and the VL is at least 90%, 91%, 92%, 93%, 94%, 95%, 96%, 97%, 98%, 99%, or 100% identical to the amino acid sequence of SEQ ID NO: 30018.

Embodiment A7 is the method of any one of the preceding embodiments, wherein the VH comprises the amino acid sequence of SEQ ID NO: 30012 and the VL comprises the amino acid sequence of SEQ ID NO: 30018.

Embodiment A8 is the method of any one of the preceding embodiments, wherein the antibody or fragment thereof is a full-length antibody.

Embodiment A9 is the method of any one of the preceding embodiments, wherein the antibody or fragment thereof comprises a fully human immunoglobulin G4 (IgG4) antibody.

Embodiment A10 is a pharmaceutical composition comprising an anti-CD39 antibody or fragment thereof and a pharmaceutically acceptable carrier, wherein the anti-CD39 antibody or fragment thereof is formulated for administration at a dose of 1400 mg or 2000 mg.

Embodiment A11 is the pharmaceutical composition of embodiment 10, wherein the anti-CD39 antibody or fragment thereof comprises:
a. six CDRs comprising:
   i. HCDR1 comprising the amino acid sequence of SEQ ID NO: 30001;
   ii. HCDR2 comprising the amino acid sequence of SEQ ID NO: 30002;
   iii. HCDR3 comprising the amino acid sequence of SEQ ID NO: 30003;
   iv. LCDR1 comprising the amino acid sequence of SEQ ID NO: 30004;
   v. LCDR2 comprising the amino acid sequence of SEQ ID NO: 30005; and
   vi. LCDR3 comprising the amino acid sequence of SEQ ID NO: 30006; and/or
b. a VH comprising the amino acid sequence of SEQ ID NO: 30012 and a VL comprising the amino acid sequence of SEQ ID NO: 30018.

Embodiment A12 is the pharmaceutical composition of embodiment 10 or embodiment 11 for use in treating cancer in a human subject in need thereof.

Embodiment A13 is the pharmaceutical composition for use of embodiment 12, wherein the anti-CD39 antibody or fragment thereof is administered intravenously.

Embodiment A14 is the pharmaceutical composition for use of embodiment 12 or embodiment 13, wherein the anti-CD39 antibody or fragment thereof is administered once every 1, 2, 3, 4, 5 or 6 weeks.

Embodiment A15 is the pharmaceutical composition for use of any one of embodiments 12-14, wherein the anti-CD39 antibody or fragment thereof is administered once every two weeks.

Embodiment A16 is the method or pharmaceutical composition for use of any one of the preceding embodiments, wherein the cancer is newly diagnosed or non-metastatic.

Embodiment A17 is the method or pharmaceutical composition for use of any one of the preceding embodiments, wherein the cancer is advanced.

Embodiment A18 is the method or pharmaceutical composition for use of any one of the preceding embodiments, wherein the cancer is refractory.

Embodiment A19 is the method or pharmaceutical composition for use of any one of the preceding embodiments, wherein the cancer is metastatic.

Embodiment A20 is the method or pharmaceutical composition for use of any one of the preceding embodiments, wherein the cancer is a solid tumor.

Embodiment A21 is the method or pharmaceutical composition for use of any one of the preceding embodiments, wherein the cancer is an advanced solid tumor.

Embodiment A22 is the method or pharmaceutical composition for use of any one of the preceding embodiments, wherein the cancer is a relapsed solid tumor.

Embodiment A23 is the method or pharmaceutical composition for use of any one of the preceding embodiments, wherein the cancer is a refractory solid tumor.

Embodiment A24 is the method or pharmaceutical composition for use of any one of the preceding embodiments, wherein the cancer is a metastatic solid tumor.

Embodiment A25 is the method or pharmaceutical composition for use of any one of the preceding embodiments, wherein the cancer is carcinoma, lymphoma, blastoma, sarcoma, or leukemia.

Embodiment A26 is the method or pharmaceutical composition for use of any one of the preceding embodiments, wherein the cancer is pancreatic cancer.

Embodiment A27 is the method or pharmaceutical composition for use of any one of the preceding embodiments, wherein the cancer is gastric cancer.

Embodiment A28 is the method or pharmaceutical composition for use of any one of the preceding embodiments, wherein the cancer is prostate cancer.

Embodiment A29 is the method or pharmaceutical composition for use of any one of the preceding embodiments, wherein the cancer is endometrial cancer.

Embodiment A30 is the method or pharmaceutical composition for use of any one of the preceding embodiments, wherein the cancer is non-small cell lung cancer.

Embodiment A31 is the method or pharmaceutical composition for use of any one of the preceding embodiments, wherein the cancer is colorectal cancer.

Embodiment A32 is the method or pharmaceutical composition for use of any one of the preceding embodiments, wherein the cancer is ovarian cancer.

Embodiment A33 is the method or pharmaceutical composition for use of any one of the preceding embodiments, wherein the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

Embodiment A34 is the method or pharmaceutical composition for use of any one of the preceding embodiments, wherein the method or use further comprises administering a second therapy.

Embodiment A35 is the method or pharmaceutical composition for use of embodiment 34, wherein the second therapy is a chemotherapeutic agent.

Embodiment A36 is the method or pharmaceutical composition for use of embodiment 34 or embodiment 35, wherein the second therapy is gemcitabine.

Embodiment A37 is the method or pharmaceutical composition for use of embodiment 34 or embodiment 35, wherein the second therapy is albumin-bound paclitaxel.

Embodiment A38 is the method or pharmaceutical composition for use of embodiment 34 or embodiment 35, wherein the second therapy is an antagonist of PD-1 or PD-L1.

Embodiment A39 is the method or pharmaceutical composition for use of embodiment 34, wherein the second therapy is an anti-PD-1 or anti-PD-L1 antibody.

Embodiment A40 is the method or pharmaceutical composition for use of any one of embodiments 1-9 or 12-33, wherein the method or use further comprises administering two additional therapies.

Embodiment A41 is the method or pharmaceutical composition for use of embodiment 40, wherein the two additional therapies comprise a chemotherapeutic agent and an antagonist of PD-1 or an antagonist of PD-L1.

Embodiment A42 is the method or pharmaceutical composition for use of embodiment 40, wherein the two additional therapies comprise a chemotherapeutic agent and an agent targeting the adenosine axis.

Embodiment A43 is the method or pharmaceutical composition for use of embodiment 40, wherein the two additional therapies comprise an antagonist of PD-1 or an antagonist of PD-L1 and an agent targeting the adenosine axis.

Embodiment A44 is the method or pharmaceutical composition for use of embodiment 40, wherein one of the two additional therapies comprise an A2AR antagonist, an A2BR antagonist, or a dual A2AR/A2B antagonist.

Embodiment A45 is the method or pharmaceutical composition for use of embodiment 40, wherein the two additional therapies comprise at least one chemotherapeutic agent.

Embodiment A46 is the method or pharmaceutical composition for use of embodiment 40, wherein the two additional therapies comprise two chemotherapeutic agents.

Embodiment A47 is the method or pharmaceutical composition for use of embodiment 45 or embodiment 46, wherein one of the two additional therapies is gemcitabine.

Embodiment A48 is the method or pharmaceutical composition for use of embodiment 45 or embodiment 46, wherein one of the two additional therapies is albumin-bound paclitaxel.

Embodiment A49 is the method or pharmaceutical composition for use of any one of embodiments 40, or 45-48, wherein the two additional therapies comprise gemcitabine and albumin-bound paclitaxel.

Embodiment A50 is the method or pharmaceutical composition for use of any one of embodiments 37, or 48-49, wherein albumin-bound paclitaxel is administered at a dose of 125 mg/m² on days 1, 8, and 15 of a 28-day cycle.

Embodiment A51 is the method or pharmaceutical composition for use of any one of embodiments 37, or 48-50, wherein albumin-bound paclitaxel is administered to the subject by intravenous administration.

Embodiment A52 is the method or pharmaceutical composition for use of any one of embodiments 36, 47, or 49, wherein gemcitabine is administered at a dose of 1000 mg/m² on days 1, 8, and 15 of a 28-day cycle.

Embodiment A53 is the method or pharmaceutical composition for use of any one of embodiments 36, 47, 49, or 52, wherein gemcitabine is administered to the subject by intravenous administration.

Embodiment A54 is the method or pharmaceutical composition for use of any one of embodiments 49-53, wherein the anti-CD39 antibody, albumin-bound paclitaxel, and gemcitabine are administered sequentially.

Embodiment A55 is the method or pharmaceutical composition for use of embodiment 54, wherein the anti-CD39 antibody is administered before albumin-bound paclitaxel and gemcitabine are administered.

Embodiment A56 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 1400 mg intravenously every 2 weeks.

Embodiment A57 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 2000 mg intravenously every 2 weeks.

Embodiment A58 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 1400 mg intravenously every 2 weeks, and further administering one or two chemotherapeutic agents.

Embodiment A59 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 2000 mg intravenously every 2 weeks, and further administering one or two chemotherapeutic agents.

Embodiment A60 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 1400 mg intravenously every 2 weeks, and further administering gemcitabine and albumin-bound paclitaxel.

Embodiment A61 is a method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 2000 mg intravenously every 2 weeks, and further administering gemcitabine and albumin-bound paclitaxel.

### BRIEF DESCRIPTION OF THE FIGURES

FIG. 1 is a schematic of a phase 1 dose-escalation study for an anti-CD39 antibody.

FIG. 2 is a swimmer plot showing individual patients' time on the study in weeks (x-axis), grouped by starting dose and their corresponding tumor type (y-axis), with the arrows indicating patients continuing therapy at the time of the data snapshot with circles denoting determination of progressive disease by RECIST criteria. Diamonds left of the (y-axis) indicate prior α-PD-(L)1 therapy. Intra-patient dose escalation was permitted, and two (2) patients were able to escalate to higher dose levels.

FIG. 3 is a waterfall plot illustrating the best percentage change in target lesions from baseline; seven (7) of nineteen (19) or 37% of patients stable disease at 8 weeks with four (4) patients or 21% persisting beyond sixteen (16) weeks.

FIG. 4 is a graphical representation of the pharmacokinetics as measured by serum concentrations after a single dose of anti-CD39 antibody administered at 20, 70, 200, 700, and 1400 mg. The anti-CD39 antibody has linear and dose-proportional pharmacokinetics.

FIG. 5 is a graphical representation of the primary pharmacodynamic marker utilized in the study subsequent to the administration of anti-CD39 antibody at doses of 20, 70, 200, 700, and 1400 mg, as measured by percent target occupancy on peripheral blood monocytes (PBMCs). The figure presents % target occupancy on the Y axis, sample timepoint on the X axis (over multiple cycles), with colors indicating different dose levels. Maximal % target occupancy was achieved at 6 hours post-dosing in patients dosed at 70 mg and above. Target Occupancy was maintained near saturation, even at trough, at doses of 200 mg and above.

FIGS. 6A-6F are baseline and week 8 images from a 60 year old man with pancreatic cancer with extensive liver metastases who received clone 22 plus gemcitabine and albumin-bound paclitaxel after progression on FOLFIRINOX chemotherapy regimen. The baseline images (FIGS. 6A, 6C and 6E) are in the top row with corresponding week 8 images (FIGS. 6B, 6D and 6F) in the bottom row, with arrows marking lesions followed for RECIST response.

FIG. 7 is a swimmer plot showing 39 individual patients' time in the monotherapy dose escalation and monotherapy tumor biopsy expansion study in weeks (x-axis), grouped by starting dose and their corresponding tumor type (y-axis), with the arrows indicating patients continuing therapy at the time of the data snapshot with circles denoting determination of progressive disease by RECIST criteria. Diamonds left of the (y-axis) indicate prior α-PD-(L)1 therapy. Intra-patient dose escalation was permitted, and two (2) patients were able to escalate to higher dose levels. From the top, rows 1-8 and 14-19 are patients who received 1400 mg doses. Rows 9-13, 20-25 are patients who received 700 mg doses. Rows 26-33 are patients who received 200 mg doses. Row 34 is a patient who received 70 mg doses from week 0 to week 16 and received 200 mg doses thereafter. Rows 35 and 36 are patients who received 70 mg doses. Row 37 is a patient who received 20 mg doses from week 0 to week 24, 70 mg doses from week 24 to week 36, and 200 mg doses from week 36 until just before week 44. Rows 38 and 39 are patients who received 20 mg doses of clone 22.

FIGS. 8A-8B are graphical summaries of the monotherapy response utilizing the Investigator Assessment per RECIST v1.1. FIG. 8A shows target lesion change over time (n=32). FIG. 8B is a waterfall plot illustrating the best percentage change in target lesions from baseline. From the left, bars 1-2, 4-5, 7-8, 10-12, and 23 are patients who received 700 mg doses. Bars 3, 13-16, 18-19, 21, 26, and 31 are patients who received 1400 mg doses. Bars 6, 9, 20, 22, 24, 25 are patients who received 200 mg doses. Bars 17, 27-28 are patients who received 70 mg doses. Bars 30 and 32 are patients who received 20 mg doses.

FIG. 9 is a swimmer plot showing 10 individual patients' time in the clone 22 and gemcitabine/albumin-bound paclitaxel combination dose escalation study in weeks (x-axis), grouped by starting dose and their corresponding tumor type (y-axis), with the arrows indicating patients continuing therapy at the time of the data snapshot with circles denoting determination of progressive disease by RECIST criteria. Diamonds left of the (y-axis) indicate no lines of prior therapy. From the top, row 1 is a patient who received a 1400 mg dose of clone 22. Rows 2-4 are patients who received 700 mg doses of clone 22. Rows 5-10 are patients who received 200 mg doses of clone 22.

FIGS. 10A-10B are graphical summaries of the combination therapy of clone 22 with gemcitabine and albumin-bound paclitaxel utilizing the Investigator Assessment per RECIST v1.1. FIG. 10B is a waterfall plot illustrating the best percentage change in target lesions from baseline. For FIG. 10A, from top to bottom, the first 5 lines are patients who received 200 mg doses of clone 22, line 5 received 700 mg of clone 22, and line 6 received 200 mg clone 22. For FIG. 10B, from the left, bars 1-5 and 7 are patients who received 200 mg doses of clone 22. Bar 6 is a patient who received 700 mg doses of clone 22.

FIG. 11 shows dose dependent loss of CD39 from circulating B cells, with sustained, complete loss starting at 700 mg.

### DETAILED DESCRIPTION OF EMBODIMENTS OF THE INVENTION

### I. Definitions

In this application, the use of “or” means “and/or” unless stated otherwise. In the context of a multiple dependent claim, the use of “or” refers back to more than one preceding independent or dependent claim in the alternative only. The terms “comprising,” “including,” and “having” can be used interchangeably herein.

The term “CD39” refers to the ectonucleoside triphosphate diphospholydrolase 1 polypeptide encoded in humans by the ENTPD1 gene. Other names for CD39 include ENTPD1, E-NTPDase1, cluster of differentiation 39, ATPDase, NTPDase-1, and SPG64.

CD39 catalyzes the hydrolysis of γ- and β-phosphate residues of extracellular nucleoside triphosphates (NTPs; e.g., adenosine triphosphate or ATP) and nucleoside diphosphates (NDPs; e.g., adenosine diphosphate or ADP), converting these molecules to the nucleoside monophosphate (NMP; e.g., adenosine monophosphate or AMP) derivative. An exemplary amino acid sequence of CD39 is at NCBI Reference Sequence: NP_001767.3.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fd, Fv, Fab, Fab′, Fab′-SH, F(ab′)2; diabodies; linear antibodies; single-chain antibody molecules (e.g. scFv); and multispecific antibodies formed from antibody fragments.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “tumor” is used herein to refer to a cell or cells that comprise a cancer. The term “tumor growth” is used herein to refer to proliferation or growth by a cell or cells that comprise a cancer that leads to a corresponding increase in the size or extent of the cancer.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

Administration “in combination with” one or more further therapeutic agents includes simultaneous (concurrent) and consecutive (sequential) administration in any order.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “dosage” refers to the amount and period of time of an administration. A “dose” refers to the amount administered. A “dosing interval” refers to the period of time between doses.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present (numbering in this paragraph is according to the EU numbering system, also called the EU index, as described in Kabat et al., Sequences of Proteins of Immunological Interest, 5^{th} Ed. Public Health Service, National Institutes of Health, Bethesda, Md., 1991).

“Framework,” “framework region,” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the human subject is referred to as a “patient.”

An increase in “interferon gamma response” or “IFNγ response” or “IFN-γ response” refers to any increase in activity associated with the cytokine IFNγ, and includes, e.g., an increase in IFNγ protein (e.g., present in the TME, secreted from a cell, or detected intracellularly), an increase in IFNγ gene expression in a cell (e.g., by measuring mRNA levels), an increase in gene expression of genes related to IFNγ (e.g., by measuring mRNA levels) (e.g., genes related to IFNγ include, e.g., Ccl8, Apoe, C1qa, C1qb, C1qc, Arg1, Lgmn, Ms4a7, Lyz2, Ccl7, Cd3g, Cxcr6, etc.). The increase may be detected in a sample from an individual after administration of a therapy e.g., as compared to a sample from an untreated individual. In some embodiments, increase may be detected in a sample from an individual after administration of a first and a second therapy e.g., as compared to a sample from an individual after administration of only the first or the second therapy.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “albumin-bound paclitaxel” is also known in the art as “nab-paclitaxel” and “Abraxane” and is paclitaxel protein-bound particles for injectable suspension (albumin-bound), formulated as albumin-bound nanoparticles.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts.

In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Wash. D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” or “pharmaceutical composition” or “pharmaceutically acceptable composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, and/or preservative.

The term “refractory,” as used herein, refers to a cancer that has not responded to a prior treatment. Refractory cancer includes a cancer that has exhibited an inadequate response to, or progressed on, a prior treatment, e.g., a prior treatment with an immuno-oncology or immunotherapy drug, e.g., with a blocking CTLA-4 or PD-1 antibody. In some embodiments, the cancer is refractory or resistant to a prior treatment, either intrinsically refractory or resistant (e.g., refractory to a PD-1 pathway antagonist), or a resistance or refractory state is acquired. The term “relapsed,” as used herein refers to a reoccurrence of a cancer in a subject. The term “metastatic,” as used herein refers to a cancer cell that has changed position from the place where it started, for example, the spread of a cancer from a primary site to another place in the body. The term “advanced,” as used herein refers to cancer that is unlikely to be cured or controlled with treatment.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, disclosed antibodies are used to delay development of a disease or to slow the progression of a disease.

As used herein, “about” or “approximately” means an acceptable error for a particular value as determined by one of ordinary skill in the art, which depends in part on how the value is measured or determined.

As used herein, numeric ranges (e.g., 1-2000) are inclusive of the numbers defining the range. Also as used herein, measured and measurable values are understood to be approximate, taking into account significant digits and the error associated with the measurement.

### II. Methods of Treating

Methods of treating cancer comprising administering an anti-CD39 antibody at particular dosages are provided. The dosages provided herein comprise a dose and a dosing interval for administration of the anti-CD39 antibody. In some embodiments, the doses are administered intravenously. It is understood that the disclosed doses include amounts that are approximately the amount of the disclosed doses.

The doses disclosed herein may be administered as a “flat” dose (or “fixed” dose) that does not depend on the weight of the subject.

In some embodiments, methods are provided for treating cancer in a human subject in need thereof comprising administering an anti-CD39 antibody, wherein the antibody is administered at a fixed or flat dose. In some embodiments, methods are provided for treating cancer in a human subject in need thereof comprising administering an anti-CD39 antibody, wherein the antibody is administered at a dose of 20, 70, 200, 700, 1400, or 2000 mg.

In some embodiments, methods are provided for treating cancer in a human subject in need thereof comprising administering an anti-CD39 antibody comprising:
a) HCDR1 comprising the amino acid sequence of SEQ ID NO: 30001;
b) HCDR2 comprising the amino acid sequence of SEQ ID NO: 30002;
c) HCDR3 comprising the amino acid sequence of SEQ ID NO: 30003;
d) LCDR1 comprising the amino acid sequence of SEQ ID NO: 30004;
e) LCDR2 comprising the amino acid sequence of SEQ ID NO: 30005; and
f) LCDR3 comprising the amino acid sequence of SEQ ID NO: 30006;
wherein the antibody is administered at a fixed or flat dose.

In some embodiments, methods are provided for treating cancer in a human subject in need thereof comprising administering an anti-CD39 antibody comprising:
a) HCDR1 comprising the amino acid sequence of SEQ ID NO: 30001;
b) HCDR2 comprising the amino acid sequence of SEQ ID NO: 30002;
c) HCDR3 comprising the amino acid sequence of SEQ ID NO: 30003;
d) LCDR1 comprising the amino acid sequence of SEQ ID NO: 30004;
e) LCDR2 comprising the amino acid sequence of SEQ ID NO: 30005; and
f) LCDR3 comprising the amino acid sequence of SEQ ID NO: 30006;
   wherein the antibody is administered at a dose of 20, 70, 200, 700, 1400, or 2000 mg.

In some embodiments, methods are provided for treating cancer in a human subject in need thereof comprising administering an anti-CD39 antibody comprising:
a) HCDR1 comprising the amino acid sequence of SEQ ID NO: 30001;
b) HCDR2 comprising the amino acid sequence of SEQ ID NO: 30002;
c) HCDR3 comprising the amino acid sequence of SEQ ID NO: 30003;
d) LCDR1 comprising the amino acid sequence of SEQ ID NO: 30004;
e) LCDR2 comprising the amino acid sequence of SEQ ID NO: 30005; and
f) LCDR3 comprising the amino acid sequence of SEQ ID NO: 30006;
   wherein the antibody is administered at a dose of 20, 70, 200, 700, 1400, or 2000 mg intravenously.

In some embodiments, methods are provided for treating cancer in a human subject in need thereof comprising administering an anti-CD39 antibody disclosed herein, wherein the antibody is administered at a dose between 20-2000 mg, 70-2000 mg, 200-2000 mg, 700-2000 mg, or 1400-2000 mg. In some embodiments, the dose is administered as a dosage once every 1, 2, 3, 4, 5 or 6 weeks. In some embodiments, the dose is administered once every 2 weeks. In some embodiments, the dose is administered intravenously.

In some embodiments, methods are provided for treating cancer in a human subject in need thereof comprising administering an anti-CD39 antibody disclosed herein, wherein the antibody is administered at a dose greater than 2000 mg. In some embodiments, the dose is administered as a dosage once every 1, 2, 3, 4, 5 or 6 weeks. In some embodiments, the dose is administered once every 2 weeks. In some embodiments, the dose is administered intravenously.

In some embodiments, methods are provided for treating cancer in a human subject in need thereof comprising administering an anti-CD39 antibody disclosed herein, wherein the antibody is administered at a dose between 20-2000 mg. In some embodiments, the antibody is administered once every 1, 2, 3, 4, 5 or 6 weeks. In some embodiments, the antibody is administered at a dose of 20 mg, once every 1 week. In some embodiments, the antibody is administered at a dose of 20 mg, once every 2 weeks. In some embodiments, the antibody is administered at a dose of 20 mg, once every 3 weeks. In some embodiments, the antibody is administered at a dose of 20 mg, once every 4 weeks. In some embodiments, the antibody is administered at a dose of 20 mg, once every 5 weeks. In some embodiments, the antibody is administered at a dose of 20 mg, once every 6 weeks. In some embodiments, the antibody is administered at a dose of 70 mg, once every 1 week. In some embodiments, the antibody is administered at a dose of 70 mg, once every 2 weeks. In some embodiments, the antibody is administered at a dose of 70 mg, once every 3 weeks. In some embodiments, the antibody is administered at a dose of 70 mg, once every 4 weeks. In some embodiments, the antibody is administered at a dose of 70 mg, once every 5 weeks. In some embodiments, the antibody is administered at a dose of 70 mg, once every 6 weeks. In some embodiments, the antibody is administered at a dose of 200 mg, once every 1 week. In some embodiments, the antibody is administered at a dose of 200 mg, once every 2 weeks. In some embodiments, the antibody is administered at a dose of 200 mg, once every 3 weeks. In some embodiments, the antibody is administered at a dose of 200 mg, once every 4 weeks. In some embodiments, the antibody is administered at a dose of 200 mg, once every 5 weeks. In some embodiments, the antibody is administered at a dose of 200 mg, once every 6 weeks. In some embodiments, the antibody is administered at a dose of 700 mg, once every 1 week. In some embodiments, the antibody is administered at a dose of 700 mg, once every 2 weeks. In some embodiments, the antibody is administered at a dose of 700 mg, once every 3 weeks. In some embodiments, the antibody is administered at a dose of 700 mg, once every 4 weeks. In some embodiments, the antibody is administered at a dose of 700 mg, once every 5 weeks. In some embodiments, the antibody is administered at a dose of 700 mg, once every 6 weeks. In some embodiments, the antibody is administered at a dose of 1400 mg, once every 1 week. In some embodiments, the antibody is administered at a dose of 1400 mg, once every 2 weeks. In some embodiments, the antibody is administered at a dose of 1400 mg, once every 3 weeks. In some embodiments, the antibody is administered at a dose of 1400 mg, once every 4 weeks. In some embodiments, the antibody is administered at a dose of 1400 mg, once every 5 weeks. In some embodiments, the antibody is administered at a dose of 1400 mg, once every 6 weeks. In some embodiments, the antibody is administered at a dose of 2000 mg, once every 1 week. In some embodiments, the antibody is administered at a dose of 2000 mg, once every 2 weeks. In some embodiments, the antibody is administered at a dose of 2000 mg, once every 3 weeks. In some embodiments, the antibody is administered at a dose of 2000 mg, once every 4 weeks. In some embodiments, the antibody is administered at a dose of 2000 mg, once every 5 weeks. In some embodiments, the antibody is administered at a dose of 2000 mg, once every 6 weeks. In some embodiments, the dose is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 20, 70, 200, 700, 1400, or 2000 mg. In some embodiments, the antibody is administered at a dose between 20-2000 mg, 70-2000 mg, 200-2000 mg, 700-2000 mg, or 1400-2000 mg. In some embodiments, the dose is administered as a dosage once every 1, 2, 3, 4, 5 or 6 weeks. In some embodiments, the dose is administered once every 2 weeks. In some embodiments, the dose is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 20 mg every 2 weeks. In some embodiments, the dosage is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 70 mg every 2 weeks. In some embodiments, the dosage is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 200 mg every 2 weeks. In some embodiments, the dosage is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 700 mg every 2 weeks. In some embodiments, the dosage is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 1400 mg every 2 weeks. In some embodiments, the dosage is administered intravenously.

In some embodiments, methods of treating cancer in a human subject in need thereof are provided comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 2000 mg every 2 weeks. In some embodiments, the dosage is administered intravenously.

In some embodiments, the dosages disclosed herein are administered intravenously.

In some embodiments, methods are provided for administering an anti-CD39 antibody to a human subject in need thereof at particular dosages wherein the administration results in enhancing, increasing and/or sustaining an anti-tumor immune response in the subject having a tumor. In some embodiments, the tumor is cancerous.

In further aspects, methods for treating cancer are provided where reducing or inhibiting the enzymatic activity of CD39 is desired comprising administering an anti-CD39 antibody as described herein at particular dosages. In some embodiments, methods for enhancing, increasing and/or sustaining an anti-tumor immune response in a subject having a tumor are provided comprising administering an anti-CD39 antibody as described herein at particular dosages. In some embodiments, the tumor is cancerous. In some embodiments, methods for treating cancer in a subject having cancer are provided comprising administering an anti-CD39 antibody as described herein at particular dosages.

In some aspects, methods are provided for alleviating one or more symptoms of cancer in a subject comprising administering an anti-CD39 antibody as described herein at particular dosages. In some aspects, methods are provided for reducing the number of symptoms or the severity of cancer in a subject comprising administering an anti-CD39 antibody as described herein at particular dosages. In a particular embodiment, the symptom of the cancer is a tumor, and a reduction is a reduction in size of a tumor, the failure of the tumor to grow, or the elimination of the tumor.

In some embodiments, methods for treating cancer are provided, comprising administering an effective amount of an anti-CD39 antibody described herein to a human subject at particular dosages. In some embodiments, the anti-CD39 antibody may inhibit the growth of at least one tumor in the subject. In some embodiments, methods for inhibiting CD39 in a tissue of a subject having cancer are provided, comprising administering the anti-CD39 antibody or composition described herein to the subject, wherein the administration reduces CD39 activity or total amount of CD39 in the tissue as compared to the activity or amount prior to administration. In some embodiments, methods of preventing CD39-mediated conversion of eATP and eADP to extracellular adenosine in a tissue of a subject having cancer are provided, comprising administering the anti-CD39 antibody described herein, wherein the administration reduces extracellular adenosine levels within the tumor microenvironment of the tissue. In some embodiments, methods of inhibiting CD39 activity in a tissue of a subject having cancer are provided, comprising administering the anti-CD39 antibody described herein, wherein the administration improves the ability to mount an immune response against a tumor cell.

Provided herein are methods for treating a human subject having cancer, comprising administering to the subject an effective amount of a CD39 antibody described herein, such that the subject is treated. A CD39 antibody can be used alone as a monotherapy. Alternatively, a CD39 antibody can be used in combination with one or more other therapies or agents, e.g., a second therapy (two total therapies), or a triple combination therapy (three total therapies), as described further below.

In some embodiments, the methods for treating a human subject having cancer by administration of an anti-CD39 antibody result in infiltration of innate immune cells into the tumor microenvironment. In some embodiments, the infiltration of innate immune cells is greater in a sample from an individual after administration of a therapy e.g., as compared to a sample from an untreated individual. In some embodiments, the infiltration of innate immune cells is greater than the infiltration of innate immune cells from administration of an antagonist of PD-1 (e.g., anti-PD-1 antibody). In some embodiments, the innate immune cells are myeloid cells. In some embodiments, the innate immune cells are tumor-associated macrophages. In some embodiments, the tumor-associated macrophages are positive for expression the F4/80 antigen. In some embodiments, the innate immune cells are NK cells.

A. Exemplary Anti-CD39 Antibodies

In some embodiments, the antibodies for use in the disclosed methods bind to and inhibit CD39. In some embodiments, the anti-CD39 antibodies reduce or inhibit the enzymatic activity of human CD39. In some embodiments, the anti-CD39 antibodies bind to recombinant CD39 and/or to membrane bound human CD39.

In some embodiments, the anti-CD39 antibodies bind to human CD39 with an equilibrium dissociation constant (KD) of less than 10 nM. In some embodiments, the anti-CD39 antibodies bind to human CD39 with a KD of about 1.11 nM. In some embodiments, the anti-CD39 antibodies bind to human CD39 and cynomolgus monkey CD39 but do not bind to mouse CD39 or rat CD39.

In some embodiments, the methods provide that the anti-CD39 antibodies inhibit or reduce conversion by human CD39 of extracellular adenosine triphosphate (eATP) or extracellular adenosine diphosphate (eADP) to extracellular adenosine monophosphate (eAMP). In some embodiments, the anti-CD39 antibodies increase the amount of eATP. In some embodiments, the anti-CD39 antibodies reduce or decrease the amount of extracellular adenosine. In some embodiments, the methods provide that the anti-CD39 antibodies maintain, increase or enhance an immunostimulatory level of eATP. In some embodiments, the anti-CD39 antibodies antagonize human CD39 in a tumor microenvironment of a tissue. In some embodiments, the methods provide that the anti-CD39 antibodies cross-react with cynomolgus CD39. In some embodiments, the methods provide that the anti-CD39 antibodies increase or enhance proliferation of a lymphocyte. In some embodiments, the methods provide that the anti-CD39 antibodies increase or enhance macrophage infiltration in tumors. In some embodiments, the methods provide that the antibodies bind to CD39 and inhibit CD39 within a normal or cancerous tissue. In some embodiments, the tissue is in the uterus, cervix, lung, prostate, breast, head, neck, colon, or ovary. In some embodiments, the tissue is in the uterus. In some embodiments, within the uterus, the antibodies inhibit CD39 in the myometrium.

The Sequence Table below provides the sequences of certain embodiments of the anti-CD39 antibodies disclosed and claimed herein.

In some embodiments, the anti-CD39 antibody for use in the disclosed methods comprises a fully human immunoglobulin G4 (IgG4) antibody.

In some embodiments, the administration of the anti-CD39 antibody reduces CD39 activity or total amount of CD39 in the tissue as compared to the activity or amount prior to administration. In some embodiments, the anti-CD39 antibody is administered at a dosage that sustains full target occupancy.

Clone 22, disclosed in the Sequence Table herein, is a fully human anti-CD39 monoclonal antibody that binds to human CD39 with nanomolar affinity and potently inhibits its enzymatic activity. Clone 22 prevents CD39-mediated conversion of ATP and adenosine diphosphate (ADP) to adenosine monophosphate (AMP) and phosphate, leading to a reduction in adenosine levels within the TME.

In some embodiments, the anti-CD39 comprises a VH comprising the HCDR1, HCDR2, and HCDR3 and a VL comprising a LCDR1, LCDR2, and LCDR3 of clone 22.

In some embodiments, the anti-CD39 antibody comprises: (a) HCDR1 comprising the amino acid sequence of SEQ ID NO: 30001; (b) HCDR2 comprising the amino acid sequence of SEQ ID NO: 30002; (c) HCDR3 comprising the amino acid sequence of SEQ ID NO: 30003; (d) LCDR1 comprising the amino acid sequence of SEQ ID NO: 30004; (e) LCDR2 comprising the amino acid sequence of SEQ ID NO: 30005; and (f) LCDR3 comprising the amino acid sequence of SEQ ID NO: 30006.

In certain embodiments, the anti-CD39 antibody comprises a VH comprising the amino acid sequence of the VH of clone 22.

In some embodiments, the anti-CD39 antibody comprises the VH of clone 22 but with 1, 2, 3, 4, or 5 amino acid substitutions outside the complementarity determining regions (CDRs), such as 1, 2, 3, 4, or 5 conservative substitutions outside the CDRs. In some embodiments, the antiCD39 antibody comprises the VH of clone 22 but with 1, 2, 3, 4, or 5 reversion substitutions outside the complementarity determining regions (CDRs).

In some embodiments, the anti-CD39 antibody comprises the VH of clone 22 but with 1, 2, 3, 4, or 5 amino acid substitutions in the framework regions of the VH sequence, such as 1, 2, 3, 4, or 5 conservative substitutions. In some embodiments, the anti-CD39 antibody comprises the VH of antibody clone 22 but with 1, 2, 3, 4, or 5 reversion substitutions in the framework regions of the VL sequence.

In some embodiments, the anti-CD39 antibody comprises the VH and VL CDRs of clone 22, wherein each CDR comprises 0, 1, 2 or 3 amino acid additions, substitutions (e.g., conservative substitutions), or deletions.

In certain embodiments, the anti-CD39 antibody comprises a VH CDR1, CDR2, and CDR3 comprising the amino acid sequences of the VH CDRs of clone 22 and comprises a VH that is at least 90%, at least 91%, at least 92%, at least 93%, at least 94%, at least 95%, at least 96%, at least 97%, at least 98%, or at least 99% identical to the VH of clone 22. In certain embodiments, the anti-CD39 antibody comprises a VH comprising an amino acid sequence that is at least 90%, at least 91%, at least 92%, at least 93%, at least 94%, at least 95%, at least 96%, at least 97%, at least 98%, or at least 99% identical to the amino acid sequence of the VH of clone 22. In certain embodiments, the VH of the anti-CD39 antibody differs from that of the VH sequences shown in the Sequence Table due to 1, 2, 3, 4, or 5 amino acid substitutions in the framework regions of the VH sequence, such as 1, 2, 3, 4, or 5 conservative substitutions. In certain embodiments, the VH of the anti-CD39 antibody differs from that of the VH sequences shown in the Sequence Table due to 1, 2, 3, 4, or 5 reversion substitutions in the framework regions of the VH sequence.

In certain embodiments, the anti-CD39 antibody comprises a VL comprising the amino acid sequence of the VL of clone 22.

In some embodiments, the-anti CD39 antibody comprises the VL of clone 22 but with 1, 2, 3, 4, or 5 amino acid substitutions outside the complementarity determining regions (CDRs), such as 1, 2, 3, 4, or 5 conservative substitutions outside the CDRs. In some embodiments, the anti-CD39 antibody comprises the VL of clone 22 but with 1, 2, 3, 4, or 5 reversion substitutions outside the complementarity determining regions (CDRs).

In some embodiments, the anti-CD39 antibody comprises the VL of clone 22 but with 1, 2, 3, 4, or 5 amino acid substitutions in the framework regions of the VL sequence, such as 1, 2, 3, 4, or 5 conservative substitutions. In some embodiments, the anti-CD39 antibody comprises the VL of antibody clone 22 but with 1, 2, 3, 4, or 5 reversion substitutions in the framework regions of the VL sequence.

In certain embodiments, the anti-CD39 antibody comprises a VL CDR1, CDR2, and CDR3 comprising the amino acid sequences of the VL CDRs of clone 22 and comprises a VL that is at least 90%, at least 91%, at least 92%, at least 93%, at least 94%, at least 95%, at least 96%, at least 97%, at least 98%, or at least 99% identical to the VL of clone 22. In certain embodiments, the anti-CD39 antibody comprises a VL comprising an amino acid sequence that is at least 90%, at least 91%, at least 92%, at least 93%, at least 94%, at least 95%, at least 96%, at least 97%, at least 98%, or at least 99% identical to the amino acid sequence of the VL of clone 22. In certain embodiments, the VL of the anti-CD39 antibody differs from that of the VL sequences shown in the Sequence Table due to 1, 2, 3, 4, or 5 amino acid substitutions in the framework regions of the VL sequence, such as 1, 2, 3, 4, or 5 conservative substitutions. In certain embodiments, the VL of the anti-CD39 antibody differs from that of the VL sequences shown in the Sequence Table due to 1, 2, 3, 4, or 5 reversion substitutions in the framework regions of the VL sequence.

In certain embodiments, the anti-CD39 antibody comprises a VH comprising the amino acid sequence of the VH of clone 22. In certain embodiments, the anti-CD39 antibody comprises a VL comprising the amino acid sequence of the VL of clone 22. In certain embodiments, the anti-CD39 antibody comprises a VH comprising the amino acid sequence of the VH of antibody clone number 22 and a VL comprising the amino acid sequence of the VL of antibody clone number 22.

In certain embodiments, the anti-CD39 antibody comprises a VH consisting of the amino acid sequence of the VH of clone 22. In certain embodiments, the anti-CD39 antibody comprises a VL consisting of the amino acid sequence of the VL of clone 22. In certain embodiments, the anti-CD39 antibody comprises a VH consisting of the amino acid sequence of the VH of antibody clone number 22 and a VL consisting of the amino acid sequence of the VL of antibody clone number 22.

In some embodiments, the anti-CD39 antibody comprises a VH comprising the VH CDRs of the VH of antibody clone number 22, and a VL comprising the VL CDRs of antibody clone number 22, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 22.

In some embodiments, the anti-CD39 antibody comprises a VH and VL, wherein the VH is at least 90%, 91%, 92%, 93%, 94%, 95%, 96%, 97%, 98%, 99%, or 100% identical to the amino acid sequence of SEQ ID NO: 30012 and the VL is at least 90%, 91%, 92%, 93%, 94%, 95%, 96%, 97%, 98%, 99%, or 100% identical to the amino acid sequence of SEQ ID NO: 30018.

In some embodiments, the anti-CD39 antibody is an IgG antibody, such as IgG1, IgG2, IgG3 or IgG4 antibody or a modified form thereof as described in the section below. In some embodiments, the constant region has effector function, and in some embodiments, the constant region is effector-less.

In certain embodiments, the anti-CD39 antibody comprises a heavy chain (HC) comprising the amino acid sequence of the heavy chain of clone 22.

In some embodiments, the anti-CD39 antibody may comprise: a heavy chain comprising the amino acid sequence of the heavy chain of antibody clone number 22 and a light chain comprising the light chain amino acid sequence of antibody clone number 22.

In some embodiments, the anti-CD39 antibody may comprise: a HC comprising the HC CDRs of the HC of antibody clone number 22, and a light chain (LC) comprising the LC CDRs of antibody clone number 22 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 22, respectively.

In some of the above embodiments, the HC and/or LC may differ from the sequence of each of the species by the presence of 1, 2, 3, 4, or 5 amino acid substitutions, such as 1, 2, 3, 4, or 5 conservative substitutions. In some of the above embodiments, the HC and/or LC may differ from the sequence of each of the species by the presence of 1, 2, 3, 4, or 5 amino acid substitutions, such as 1, 2, 3, 4, or 5 reversion substitutions.

In certain embodiments, the anti-CD39 antibody is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Antibody fragments can be made by various techniques, including but not limited to proteolytic digestion of an intact antibody as well as production by recombinant host cells (e.g. E. coli or phage), as described herein.

B. Recombinant Methods

Antibodies may be produced using recombinant methods and compositions, e.g., as described in U.S. Pat. No. 4,816,567, or as described in WO2019178269. In some embodiments, an isolated nucleic acid(s) encoding an anti-CD39 antibody described herein is provided. Such nucleic acid(s) may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid(s) are provided. In a further embodiment, a host cell comprising such nucleic acid(s) is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g. a Chinese Hamster Ovary (CHO) cell or lymphoid cell (e.g., Y0, NS0, Sp20 cell). In some embodiments, a method of making an anti-CD39 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD39 antibody, a nucleic acid(s) encoding an antibody, e.g., as described above, is isolated and inserted into one or more vectors for further cloning and/or expression in a host cell. Such nucleic acid(s) may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Pharmaceutical Compositions

In some embodiments, pharmaceutical compositions are provided comprising an anti-CD39 antibody and a pharmaceutically acceptable carrier, wherein the anti-CD39 antibody is formulated for administration at a dose of 20, 70, 200, 700, 1400, or 2000 mg. In some embodiments, pharmaceutical compositions are provided comprising an anti-CD39 antibody comprising:
(a) six CDRs comprising:
   i. HCDR1 comprising the amino acid sequence of SEQ ID NO: 30001;
   ii. HCDR2 comprising the amino acid sequence of SEQ ID NO: 30002;
   iii. HCDR3 comprising the amino acid sequence of SEQ ID NO: 30003;
   iv. LCDR1 comprising the amino acid sequence of SEQ ID NO: 30004;
   v. LCDR2 comprising the amino acid sequence of SEQ ID NO: 30005; and
   vi. LCDR3 comprising the amino acid sequence of SEQ ID NO: 30006; or
(b) a VH comprising the amino acid sequence of SEQ ID NO: 30012 and a VL comprises the amino acid sequence of SEQ ID NO: 30018;
   and a pharmaceutical carrier, wherein the anti-CD39 antibody is formulated for administration at a dose of 20, 70, 200, 700, 1400, or 2000 mg.

In some embodiments, the pharmaceutical composition is for use in treating cancer in a human subject in need thereof. In some embodiments of the pharmaceutical composition, the anti-CD39 antibody is administered intravenously. In some embodiments, the anti-CD39 antibody is administered once every 1, 2, 3, 4, 5 or 6 weeks. In some embodiments, the anti-CD39 antibody is administered once every two weeks.

D. Treatment Measures

In some embodiments, results of the treatment methods provided herein are evaluated. In some embodiments, disease progression of the subject having cancer may evaluated using known response evaluation criteria (e.g., Response Evaluation Criteria in Solid Tumors (RECIST)). In some embodiments, response to treatment is evaluated using tumor biopsies. In some embodiments, response to treatment is evaluated using a CT scan of chest, abdomen, pelvis, fluorodeoxyglucose-positron emission tomography, and/or magnetic resonance imaging. In some embodiments, response to treatment is evaluated by measuring antidrug antibodies, electrocardiograms (ECGs), and/or safety laboratory values. In some embodiments, response to treatment is evaluated by measuring levels of anti-CD39 antibody target occupancy and/or serum concentrations of anti-CD39 antibody. In some embodiments, response to treatment is evaluated by measuring levels of intratumoral CD39 enzymatic activity. In some embodiments, the objective response rate (ORR), duration of response (DoR), disease control rate (DCR), progression-free survival (PFS), and landmark PFS rate are determined. In some embodiments, response to treatment is evaluated by measuring changes in selected blood, serum/plasma, cytokines, and/or tumor tissue biomarkers, which may include gene and protein expression levels, tumor DNA mutation and copy number variations, and immune cell population subset enumeration and evaluation. In some embodiments, response to treatment is evaluated by measuring germline DNA polymorphic sequence variations in relation to the PK, pharmacodynamics, safety, and/or preliminary efficacy of the anti-CD39 antibody. In some embodiments, response to treatment is evaluated by measuring serum concentrations of the combination therapy agents.

E. Diseases and Disorders

In the methods for treating cancer provided herein, cancers can be cancers with solid tumors or blood malignancies (e.g., liquid tumors). In some embodiments, the cancer is newly diagnosed. In some embodiments, the cancer is non-metastatic. In some embodiments, the cancer is advanced. In some embodiments, the cancer is relapsed. In some embodiments, the cancer is refractory. In some embodiments, the cancer is metastatic. In some embodiments, the cancer is a solid tumor. In some embodiments, the cancer is an advanced solid tumor. In some embodiments, the cancer is a relapsed solid tumor. In some embodiments the cancer is a refractory solid tumor. In some embodiments, the cancer is a metastatic solid tumor. In some embodiments, the cancer is an advanced, relapsed solid tumor. In some embodiments, the cancer is an advanced, refractory solid tumor. In some embodiments, the cancer is an advanced, metastatic solid tumor. In some embodiments, the cancer is a relapsed, refractory solid tumor. In some embodiments, the cancer is a relapsed, metastatic solid tumor. In some embodiments, the cancer is a refractory, metastatic tumor. In some embodiments, the tumor is an advanced, relapsed, refractory solid tumor. In some embodiments, the cancer is an advanced, relapsed, metastatic tumor. In some embodiments, the cancer is an advanced, refractory, metastatic tumor. In some embodiments, the cancer is a relapsed, refractory, metastatic solid tumor. In some embodiments, the tumor is an advanced, relapsed, refractory, metastatic solid tumor.

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, hepatocellular carcinoma (HCC), colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, sarcoma, sinonasal natural killer, melanoma (e.g., malignant melanoma, such as cutaneous or intraocular malignant melanoma and metastatic malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, angiosarcoma, spinal axis tumor, brain cancer, brain stem glioma, pituitary adenoma, Kaposi's sarcoma, epidermoid cancer, squamous cell cancer, T cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B cell lymphomas, T cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T cell lymphoma/leukemia, mantle cell lymphoma, angio immunoblastic T cell lymphoma, angiocentric lymphoma, intestinal T cell lymphoma, primary mediastinal B-cell lymphoma, precursor T-lymphoblastic lymphoma, T-lymphoblastic; and lymphoma/leukaemia (T-Lbly/T-ALL), peripheral T cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous system, including astrocytoma, oligodendroglioma, medulloblastoma, peripheral nerve sheath tumors, PNET, schwannomas; tumors of mesenchymal origin, including fibrosarcoma, rhabdomyoscaroma, and osteosarcoma; and other tumors, including xeroderma pigmentosum, keratoacanthoma, seminoma, thyroid follicular cancer and teratocarcinoma, hematopoietic tumors of lymphoid lineage, for example T cell and B cell tumors, including but not limited to T cell disorders such as T-prolymphocytic leukemia (T-PLL), including of the small cell and cerebriform cell type; large granular lymphocyte leukemia (LGL) of the T cell type; a/d T-NHL hepatosplenic lymphoma; peripheral/post-thymic T cell lymphoma (pleomorphic and immunoblastic subtypes); angiocentric (nasal) T cell lymphoma; cancer of the head or neck, renal cancer, rectal cancer, cancer of the thyroid gland; acute myeloid lymphoma, as well as any combinations of said cancers. The methods described herein can be used for treatment of metastatic cancers, and/or unresectable cancers, and/or relapsed cancers, and/or refractory cancers, and/or advanced cancers, and/or recurrent cancers. The methods described herein can be used for treatment of pancreatic cancer. The methods described herein can be used for treatment of gastric cancer.

The methods described herein can be used for treatment of prostate cancer. The methods described herein can be used for treatment of endometrial cancer. The methods described herein can be used for treatment of non-small cell lung cancer. The methods described herein can be used for treatment of colorectal cancer. The methods described herein can be used for the treatment of ovarian cancer.

In certain embodiments, an antibody described herein is administered to subjects having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist or other immune checkpoint therapy including CTLA4 inhibitors), or a resistance or refractory state is acquired. For example, an antibody described herein may be administered to subjects who are not responsive or not sufficiently responsive to a first therapy or who have disease progression following treatment, e.g., immune checkpoint therapies including anti-PD-1 pathway antagonist treatment or CTLA4 inhibitors, either alone or in combination with another therapy (e.g., with an anti-PD-1 pathway antagonist therapy). In other embodiments, an antibody described herein is administered to subjects who have not previously received (i.e., been treated with) an immuno-oncology agent, e.g., a PD-1 pathway antagonist.

F. Combinations

The methods of treating cancer provided herein may comprise administering an anti-CD39 antibody at particular doses and dosages disclosed herein in combination with one or more additional therapeutic agents. In some embodiments, the anti-CD39 antibody is administered with one additional therapy or agent (e.g., also sometimes referred to herein as a second therapy). In some embodiments, the anti-CD39 antibody may be used in combination with at least one additional therapeutic agent (e.g., further comprising administering a second therapy, or further comprising administering a second therapy and a third therapy (a triple-combination of cancer therapies) or further comprising administering a second therapy, a third therapy and a fourth therapy (a quadruple combination of cancer therapies)).

In some embodiments, targeting an additional independent inhibitory pathway or combinations thereof has the potential to lead to further enhanced immune cell activation beyond monotherapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy and a third therapy.

In some embodiments, methods are provided for treating cancer in a subject comprising administering an anti-CD39 antibody to a subject having cancer at particular doses and dosages disclosed herein, wherein the method further comprises administering a second therapy. In some embodiments, methods are provided for treating cancer in a subject comprising administering an anti-CD39 antibody to a subject having cancer at particular doses and dosages disclosed herein, wherein the method further comprises administering a second therapy and a third therapy. In some embodiments, methods are provided for inhibiting CD39 in tissue of a subject having cancer comprising administering an anti-CD39 antibody to a subject having cancer at particular doses and dosages disclosed herein, wherein the administration reduces CD39 activity or total amount of CD39 in the tissue as compared to the activity or amount prior to administration, and wherein the method further comprises administering a second therapy. In some embodiments, methods are provided for inhibiting CD39 in tissue of a subject having cancer comprising administering an anti-CD39 antibody to a subject having cancer at particular doses and dosages disclosed herein, wherein the administration reduces CD39 activity or total amount of CD39 in the tissue as compared to the activity or amount prior to administration, and wherein the method further comprises administering a second therapy and a third therapy.

In some embodiments, methods are provided for enhancing, increasing and/or sustaining an anti-tumor immune response in a subject comprising administering an anti-CD39 antibody to a subject having a tumor at particular doses and dosages disclosed herein, wherein the method further comprises administering a second therapy, wherein the second therapy is an antagonist of PD-1 or PD-L1. In some embodiments, methods are provided for enhancing, increasing and/or sustaining an anti-tumor immune response in a subject comprising administering an anti-CD39 antibody to a subject having a tumor at particular doses and dosages disclosed herein, wherein the method further comprises administering an antagonist of PD-1 or PD-L1 and a third therapy that is different from the first and second.

In some embodiments, methods are provided for treating cancer in a subject comprising administering an anti-CD39 antibody to a subject having cancer at particular doses and dosages disclosed herein, wherein the method further comprises administering a second therapy, wherein the second therapy is an antagonist of PD-1 or PD-L1. In some embodiments, methods are provided for treating cancer in a subject comprising administering an anti-CD39 antibody to a subject having cancer at particular dosages disclosed herein, wherein the method further comprises administering an antagonist of PD-1 or PD-L1 and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an antagonist of PD-1 or PD-L1. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an antagonist of PD-1 or PD-L1 and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy is an antagonist of PD-Li. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an antagonist of PD-L1 and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy is an antagonist of PD-L1. In some embodiments, methods are provided for treating cancer in a subject comprising administering an anti-CD39 antibody to a subject having cancer at particular dosages disclosed herein, wherein the method further comprises administering an antagonist of PD-L1 and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an antagonist of PD-L1. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an antagonist of PD-L1 and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy is an antagonist of CD73. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an antagonist of CD73 and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy is an antagonist of CD73. In some embodiments, methods are provided for treating cancer in a subject comprising administering an anti-CD39 antibody to a subject having cancer at particular dosages disclosed herein, wherein the method further comprises administering an antagonist of CD73 and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an antagonist of CD73. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an antagonist of CD73 and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy is an A2AR antagonist, an A2BR antagonist, or a dual A2AR/A2B antagonist. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an A2AR antagonist, an A2BR antagonist, or a dual A2AR/A2B antagonist and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy is an A2AR antagonist, an A2BR antagonist, or a dual A2AR/A2B antagonist. In some embodiments, methods are provided for treating cancer in a subject comprising administering an anti-CD39 antibody to a subject having cancer at particular dosages disclosed herein, wherein the method further comprises administering an A2AR antagonist, an A2BR antagonist, or a dual A2AR/A2B antagonist and a third therapy. In some embodiments, methods are provided for treating cancer in a subject comprising administering an anti-CD39 antibody comprising:
a) HCDR1 comprising the amino acid sequence of SEQ ID NO: 30001;
b) HCDR2 comprising the amino acid sequence of SEQ ID NO: 30002;
c) HCDR3 comprising the amino acid sequence of SEQ ID NO: 30003;
d) LCDR1 comprising the amino acid sequence of SEQ ID NO: 30004;
e) LCDR2 comprising the amino acid sequence of SEQ ID NO: 30005; and
f) LCDR3 comprising the amino acid sequence of SEQ ID NO: 30006;
   wherein the antibody is administered at a dose of 20, 70, 200, 700, 1400, or 2000 mg, and wherein the method further comprises administering an A2AR antagonist, an A2BR antagonist, or a dual A2AR/A2B antagonist, and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an A2AR antagonist, an A2BR antagonist, or a dual A2AR/A2B antagonist. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an A2AR antagonist, an A2BR antagonist, or a dual A2AR/A2B antagonist and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy is an antagonist of CD47. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an antagonist of CD47 and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy is an antagonist of CD47. In some embodiments, methods are provided for treating cancer in a subject comprising administering an anti-CD39 antibody to a subject having cancer at particular dosages disclosed herein, wherein the method further comprises administering an antagonist of CD47 and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an antagonist of CD47. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an antagonist of CD47 and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy modulates IL-27 signaling. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an agent that modulates IL-27 signaling and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy modulates IL-27 signaling. In some embodiments, methods are provided for treating cancer in a subject comprising administering an anti-CD39 antibody to a subject having cancer at particular dosages disclosed herein, wherein the method further comprises administering an agent that modulates IL-27 signaling and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy modulates IL-27 signaling. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an agent that modulates IL-27 signaling and a third therapy.

In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering a second therapy, wherein the second therapy is an antagonist of CTLA4. In some embodiments, methods are provided for enhancing, increasing and/or sustaining an anti-tumor immune response in a subject comprising administering an anti-CD39 antibody to a subject having a tumor at particular dosages disclosed herein, wherein the method further comprises administering an antagonist of CTLA4 and a third therapy.

In some embodiments, methods are provided for treating cancer in a subject comprising administering an anti-CD39 antibody to a subject having cancer at particular dosages disclosed herein, wherein the method further comprises administering a second therapy, wherein the second therapy is an antagonist of CTLA4. In some embodiments, methods are provided for treating cancer in a subject comprising administering an anti-CD39 antibody to a subject having cancer at particular dosages disclosed herein, wherein the method further comprises administering an antagonist of CTLA4 and a third therapy.

In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering a second therapy, wherein the second therapy is an antagonist of CTLA4. In some embodiments, methods are provided for inhibiting CD39 in tissue of a subject having cancer comprising administering an anti-CD39 antibody to a subject having cancer at particular dosages disclosed herein, wherein the administration reduces CD39 activity or total amount of CD39 in the tissue as compared to the activity or amount prior to administration, and wherein the method further comprises administering an antagonist of CTLA4 and a third therapy.

In some embodiments, the additional therapeutic agent or second therapy or third therapy is a chemotherapeutic agent, an opsonizing agent, a regulatory T cell (“Treg”) depleting agent, an antagonist of a target other than CD39, or an agonist of a target other than CD39. In certain embodiments, the additional therapeutic agent or second therapy or third therapy is a chemotherapeutic agent described herein or any known chemotherapeutic agent. In some embodiments, the additional therapeutic agent or second therapy or third therapy is an opsonizing agent, wherein the opsonizing agent is an antibody other than an anti-CD39 antibody that targets cancer or tumor cells. In some embodiments, the additional therapeutic agent or second therapy or third therapy is a Treg depleting agent described herein or any known Treg depleting agent. In some embodiments, the additional therapeutic agent or second therapy or third therapy is an antagonist of a target other than CD39. In some embodiments, the additional therapeutic agent or second therapy or third therapy is an agonist of a target other than CD39.

In some instances, the additional therapeutic agent or second therapy or third therapy targets an independent inhibitory pathway, such as, for example, a pathway involving PD-1, PD-L1, CTLA-4, Lag-3, TIM-3, A2AR, A2BR, CD40, TIGIT, CD112R or CD73. In some embodiments, the additional therapeutic agent or second therapy or third therapy antagonizes one or more of PD-1, PD-L1, CTLA-4, Lag-3, TIM-3, A2AR, A2BR, CD40, TIGIT, CD112R or CD73. In some embodiments, the additional therapeutic agent or second therapy or third therapy is an agent targeting the adenosine axis. In some embodiments, the agent targeting the adenosine axis is a CD73 inhibitor. In some embodiments, the agent targeting the adenosine axis is an A2AR, A2BR or dual A2AR/A2BR antagonist. Suitable antagonists for use in the combination therapy described herein, include, without limitation, ligands, antibodies (e.g., monoclonal antibodies and bispecific antibodies), and multivalent agents. In one embodiment, the antagonist is a fusion protein, e.g., an Fc fusion protein, such as AMP-244. In some embodiments, the PD-1 or PD-L1 antagonist is an anti-PD-1 or anti-PD-L1 antibody.

In some embodiments, the methods further comprise administering a second therapy, wherein the second therapy is administration of an antagonist of PD-1 or PD-L1 (e.g., anti-PD-1 or anti-PD-L1 antibody). In some embodiments, the methods result in an increase in interferon gamma (IFN-γ) response in the tumor microenvironment. In some embodiments, the increase in IFN-γ response is greater than the IFN-γ response from administration of the antagonist of PD-1 or PD-L1 alone. In some embodiments, the increase in IFN-γ response is greater than the IFN-γ response from a subject that has not received administration of the antagonist of PD-1 or PD-L1. In some embodiments, the increase in IFN-γ response is upregulation of interferon gamma (IFN-γ)-related genes in tumor-associated macrophages in the tumor microenvironment. In some embodiments, the increase in IFN-γ response is an increase in IFN-γ protein amount in the tumor microenvironment. In some embodiments, the increase in IFN-γ response is an increase in IFN-γ gene expression in cells isolated from the tumor microenvironment.

In some embodiments, the methods for treating a subject having cancer with an anti-CD39 antibody at particular dosages disclosed herein and an antagonist of PD-1 or PD-L1 (e.g., anti-PD-1 or anti-PD-L1 antibody) result in infiltration of innate immune cells into the tumor microenvironment. In some embodiments, the infiltration of innate immune cells is greater than the infiltration of innate immune cells from administration of the antagonist of PD-1 or PD-L1 alone. In some embodiments, the infiltration of innate immune cells is greater than the infiltration of innate immune cells from a subject that has not received administration of the antagonist of PD-1 or PD-L1. In some embodiments, the innate immune cells are myeloid cells. In some embodiments, the innate immune cells are tumor-associated macrophages. In some embodiments, the tumor-associated macrophages are positive for expression the F4/80 antigen. In some embodiments, the innate immune cells are NK cells.

An exemplary anti-PD-1 antibody is nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is AMP-514 described in WO 2012/145493; PDR001; BGB-A317 (tislelizumab) and BGB-108; 244C8 and 388D4 as described in WO2016106159; REGN2810; pidilizumab; TSR-042; PF-06801591; or AMP-224. Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO2013/173223 can also be used. An anti-PD-1 antibody that competes for binding with, and/or binds to the same epitope on PD-1 as, as one of these antibodies can also be used in combination treatments.

In some embodiments, the anti-PD-L1 antibody useful for the combination therapy is BMS-936559 (referred to as 12A4 in WO 2007/005874 and U.S. Pat. No. 7,943,743), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MED14736 (also known as durvalumab and Anti-B7-H1), MPDL3280A (also known as atezolizumab and RG7446), MSB0010718C (also known as avelumab; WO2013/79174), FAZ053, MDX1105, or rHigM12B7. Any of the anti-PD-L1 antibodies disclosed in WO2013/173223, WO2011/066389, WO2012/145493, U.S. Pat. Nos. 7,635,757 and 8,217,149 and U.S. Publication No. 2009/145493 can also be used. Anti-PD-L1 antibodies that compete with and/or bind to the same epitope as that of any of these antibodies can also be used in combination treatments.

In some embodiments of the method of treating cancer involving administering a second therapy, the second therapy is an antagonist of PD-1. In some embodiments of the method of treating cancer involving administering a second therapy, the second therapy is an antagonist of PD-L1. In some embodiments, the antagonist of PD-1 is an anti-PD-1 antibody. In some embodiments, the antagonist of PD-L1 is an anti-PD-L1 antibody. In some embodiments, the antagonist of PD-1 is administered to the subject by intravenous administration. In some embodiments, the anti-CD39 antibody and the antagonist of PD-1 are administered sequentially. In some embodiments, the antagonist of PD-1 is administered before the anti-CD39 antibody is administered.

In some embodiments of the method of treating cancer, the method further comprises administering two additional therapies. In some embodiments the two additional therapies comprise a chemotherapeutic agent and an antagonist of PD-1 or an antagonist of PD-L1. In some embodiments, the two additional therapies comprise a chemotherapeutic agent and an agent targeting the adenosine axis. In some embodiments, the two additional therapies comprise an antagonist of PD-1 or an antagonist of PD-L1 and an agent targeting the adenosine axis.

In some embodiments, methods of treating cancer in a subject in need thereof are provided comprising administering an anti-CD39 antibody comprising disclosed herein, wherein the antibody is administered at a dose of 20, 70, 200, 700, 1400, or 2000 mg, and wherein the method further comprises administering a second therapy. In some embodiments, the second therapy is a chemotherapeutic agent. In some embodiments, the second therapy is gemcitabine. In some embodiments, the second therapy is albumin-bound paclitaxel. In some embodiments, the second therapy is an antagonist of PD-1. In some embodiments, the second therapy is an anti-PD-1 antibody. In some embodiments, the anti-CD39 antibody is administered intravenously. In some embodiments, the anti-CD39 antibody is administered once every 1, 2, 3, 4, 5 or 6 weeks. In some embodiments, the anti-CD39 antibody is administered once every 2 weeks.

In some embodiments, methods of treating cancer in a subject in need thereof are provided comprising administering an anti-CD39 antibody disclosed herein, wherein the antibody is administered at a fixed or flat dose, and wherein the method further comprises administering an anti-PD-1 antibody. In some embodiments, methods of treating cancer in a subject in need thereof are provided comprising administering an anti-CD39 antibody disclosed herein, wherein the antibody is administered at a dose of 20, 70, 200, 700, 1400, or 2000 mg, and wherein the method further comprises administering an anti-PD-1 antibody. In some embodiments, a method of treating cancer in a subject in need thereof are provided comprising administering an anti-CD39 antibody comprising:
g) HCDR1 comprising the amino acid sequence of SEQ ID NO: 30001;
h) HCDR2 comprising the amino acid sequence of SEQ ID NO: 30002;
i) HCDR3 comprising the amino acid sequence of SEQ ID NO: 30003;
j) LCDR1 comprising the amino acid sequence of SEQ ID NO: 30004;
k) LCDR2 comprising the amino acid sequence of SEQ ID NO: 30005; and
1) LCDR3 comprising the amino acid sequence of SEQ ID NO: 30006;
   wherein the antibody is administered at a dose of 20, 70, 200, 700, 1400, or 2000 mg, and wherein the method further comprises administering an anti-PD-1 antibody. In some embodiments, the anti-CD39 antibody is administered intravenously. In some embodiments, the anti-CD39 antibody is administered once every 1, 2, 3, 4, 5 or 6 weeks. In some embodiments, the anti-CD39 antibody is administered once every 2 weeks. In some embodiments, the anti-CD39 antibody and anti-PD-1 antibody are administered sequentially. In some embodiments, the anti-PD-1 antibody is administered before the anti-CD39 antibody is administered.

In some embodiments, methods of treating cancer in a subject in need thereof are provided comprising administering an anti-CD39 antibody comprising disclosed herein, wherein the antibody is administered at a fixed or flat dose, and wherein the method further comprises administering gemcitabine and albumin-bound paclitaxel. at a fixed or flat dose. In some embodiments, methods of treating cancer in a subject in need thereof are provided comprising administering an anti-CD39 antibody comprising disclosed herein, wherein the antibody is administered at a dose of 20, 70, 200, 700, 1400, or 2000 mg, and wherein the method further comprises administering gemcitabine and albumin-bound paclitaxel. In some embodiments, a method of treating cancer in a subject in need thereof are provided comprising administering an anti-CD39 antibody comprising:
a) HCDR1 comprising the amino acid sequence of SEQ ID NO: 30001;
b) HCDR2 comprising the amino acid sequence of SEQ ID NO: 30002;
c) HCDR3 comprising the amino acid sequence of SEQ ID NO: 30003;
d) LCDR1 comprising the amino acid sequence of SEQ ID NO: 30004;
e) LCDR2 comprising the amino acid sequence of SEQ ID NO: 30005; and
f) LCDR3 comprising the amino acid sequence of SEQ ID NO: 30006;
   wherein the antibody is administered at a dose of 20, 70, 200, 700, 1400, or 2000 mg, and wherein the method further comprises administering gemcitabine and albumin-bound paclitaxel. In some embodiments, the albumin-bound paclitaxel is administered at a dose of 125 mg/m² on days 1, 8, and 15 of a 28-day cycle. In some embodiments, the albumin-bound paclitaxel is administered to the subject by intravenous administration. In some embodiments, the gemcitabine is administered at a dose of 1000 mg/m² on days 1, 8, and 15 of a 28-day cycle. In some embodiments, the gemcitabine is administered to the subject by intravenous administration. In some embodiments, the anti-CD39 antibody is administered intravenously. In some embodiments, the anti-CD39 antibody is administered once every 1, 2, 3, 4, 5 or 6 weeks. In some embodiments, the anti-CD39 antibody is administered once every 2 weeks. In some embodiments, the anti-CD39 antibody, albumin-bound paclitaxel, and gemcitabine are administered sequentially. In some embodiments, the anti-CD39 antibody is administered before albumin-bound paclitaxel and gemcitabine are administered.

In certain embodiments, the methods may comprise administering an anti-CD39 antibody in combination with a CTLA-4 antagonist, e.g., an anti-CTLA-4 antibody. In one embodiment, an anti-CTLA-4 antibody is an antibody selected from the group of: Yervoy® (ipilimumab or antibody 10D1, described in PCT Publication WO 01/14424), tremelimumab (formerly ticilimumab, CP-675,206), monoclonal or an anti-CTLA-4 antibody described in any of the following publications: WO 98/42752; WO 00/37504; U.S. Pat. No. 6,207,156; Hurwitz et al. (1998) Pro. Natl. Acad. Sci. USA 95(17): 10067-10071; Camacho et al. (2004) J. Clin. Oncology 22(145): antibodies tract No. 2505 (antibody CP-675206); and Mokyr et al. (1998) Cancer Res. 58:5301-5304. Any of the anti-CTLA-4 antibodies disclosed in WO2013/173223 can also be used.

In some embodiments, the methods may comprise administering an anti-CD39 antibody in combination with a LAG-3 (also referred to herein and by others as LAG3) antagonist. In some embodiments, the LAG-3 inhibitor is selected from the group consisting of LAG525, BMS-986016, and TSR-033. Examples of anti-LAG3 antibodies include antibodies comprising the CDRs or variable regions of antibodies 25F7, 26H10, 25E3, 8B7, 11F2 or 17E5, which are described in U.S. Patent Publication No. US2011/0150892, WO10/19570 and WO2014/008218. In one embodiment, an anti-LAG-3 antibody is BMS-986016. Other art recognized anti-LAG-3 antibodies that can be used include IMP731 and IMP-321, described in US 2011/007023, WO08/132601, and WO09/44273. Anti-LAG-3 antibodies that compete with and/or bind to the same epitope as that of any of these antibodies can also be used in combination treatments.

In some embodiments, the methods may comprise administering an anti-CD39 antibody in combination with an adenosine A2AR antagonist, A2BR antagonist or dual A2AR/A2BR antagonists. Examples of A2AR, A2BR and dual A2AR/A2BR antagonists include Preladenant/SCH 420814 (Merck/Schering, CAS Registry Number: 377727-87-2), which is described in Hodgson et al., (2009) J Pharmacol Exp Ther 330(1):294-303 and incorporated herein by reference in its entirety; ST-4206 (Leadiant Biosciences), which is described in U.S. Pat. No. 9,133,197 and incorporated herein by reference in its entirety; KW-6356 (Kyowa Hakko Kogyo), Tozadenant/SYN-115 (Acorda), Istradefylline/KW-6002 (Kyowa Hakko Kogyo, CAS Registry Number: 155270-99-8), which is described in LeWitt et al., (2008) Ann Neurol 63(3):295-302 and is incorporated herein by reference in its entirety; theophylline (CAS Registry Number: 58-55-9), NIR178 (Novartis); GBV-2034 (Globavir), Vipadenant (Redox/Juno), AZD4635/HTL-1071 (AstraZeneca/Heptares), which is described in WO2011/095625 and is incorporated herein by reference in its entirety; CPI-444/V81444 (Corvus/Genentech), which is described in WO 2009/156737 and is incorporated herein by reference in its entirety; PBF509 (Palobiofarma/Novartis), which is described in U.S. Pat. No. 8,796,284 and WO 2017/025918 and are incorporated herein by reference in their entirety; A2AR antagonists described in U.S. Pat. Nos. 8,114,845, 9,029,393, US20170015758, or US20160129108, all of which are incorporated herein by reference in their entirety; and ATL-444, MSX-3, SCH-58261, SCH-412,348, SCH-442,416, VER-6623, VER-6947, VER-7835, CGS-15943, or ZM-241,385.

In some embodiments, the methods may comprise administering an anti-CD39 antibody in combination with an adenosine A2BR antagonist. In some embodiments, the methods may comprise administering an anti-CD39 antibody in combination with a dual A2AR/A2BR antagonist.

In some embodiments, the methods may comprise administering an anti-CD39 antibody in combination with a CD40 inhibitor.

In some embodiments, the methods may comprise administering an anti-CD39 antibody in combination with an agent targeting the adenosine axis (e.g., a CD73 inhibitor or a A2AR/A2BR antagonist).

In some embodiments, the methods may comprise administering an anti-CD39 antibody in combination with a CD73 inhibitor. Examples of CD73 inhibitors include small molecule CD73 inhibitors such as AB421 (Arcus), a CD73 antibody, or antigen binding portion thereof, that binds to CD73 such as MED19447 (Medimmune), BMS-986179 (Bristol Meyers Squibb), or such as described in US2018/0009899 (Corvus), which is incorporated herein by reference in its entirety.

In some embodiments, the methods may comprise administering an anti-CD39 antibody in combination with a TIM-3 inhibitor. Examples of TIM-3 inhibitors include MGB453 (Novartis), TSR-022 (Tesaro), or LY3321367 (Eli Lilly). Suitable antagonists for use in the combination therapy described herein, include, without limitation, ligands, antibodies (e.g., monoclonal antibodies and bispecific antibodies), and multivalent agents.

In some embodiments, the one or more additional therapeutic agents is a chimeric antigen receptor (CAR) cell therapy. In some embodiments, the CAR cell therapy is CTL019.

In some embodiments, members of the PVR gene family are upregulated on tumor cells and can exhibit intrinsic tumor-promoting properties. Therefore, in some embodiments, the second therapy is selected from one or more of an antagonist of TIGIT, CD112R, CD96, PVRL1, PVRL2, PVRL3, PVRL4, and CD155. Suitable antagonists for use in the combination therapy described herein, include, without limitation, ligands, antibodies (e.g., monoclonal antibodies and bispecific antibodies), and multivalent agents.

STING agonists induce innate immune cell activation resulting in increased T cell priming and recruitment of immune cells into the tumor microenvironment. Targeting STING agonists in combination with CD39 has the potential to lead to an even further increase in T cell and NK cell recruitment and activation.

Increased anti-CD47 antibody mediated phagocytosis can lead to an increase in the presentation of cancer derived antigens by macrophages to T cells. Combination treatment with an anti-CD47 antibody and an anti-CD39 antibody, such as an anti-CD39 antibody provided herein provides an opportunity to enhance cancer antigen specific T cell responses and is fully encompassed herein.

In some embodiments, the additional therapeutic agent or second therapy or third therapy is an antagonist of CD47. In some embodiments, the antagonist of CD47 is an anti-CD47 antibody. See U.S. Pat. No. 9,803,016 (e.g., SEQ Id NOs: 24 and 26), herein incorporated in its entirety by reference.

Any of the therapies listed above for use in combination with an anti-CD39 antibody at fixed doses may be used alone with the anti-CD39 antibody or together with any of the other named non-anti-CD39 therapy.

The methods disclosed herein may also be provided before, substantially contemporaneous with, or after other therapies, for example, surgery, chemotherapy, radiation therapy, or the administration of a biologic, such as another therapeutic antibody. In some embodiments where e.g., two therapies are administered or a triple-combination of cancer therapies is administered, the therapies may be administered concurrently, consecutively, and/or at different points in time according to their own dosing schedule. In some embodiments, the cancer has recurred or progressed following a therapy selected from surgery, chemotherapy, and radiation therapy, or a combination thereof. For example, the methods described herein could be provided as adjunctive therapy when there is a risk that micrometastases can be present and/or in order to reduce the risk of a relapse.

In some embodiments, the methods provided herein comprise administering a anti-CD39 antibody at particular doses and dosages disclosed herein and a chemotherapeutic agent. Exemplary chemotherapeutic agents include, but are not limited to, anthracyclines (e.g., doxorubicin, idarubicin, daunorubicin, cytarabine, epirubicin, valrubicin and mitoxantrone) (see e.g., Minotti et al., (2004) Pharmacol Rev 56(2):185-229), topoisomerase inhibitors (e.g., topotecan; Hycamtin, camptothecin, etoposide) (see e.g., Pommier et al., (2010) Chem Biol 17(5):421-433; which is incorporated herein by reference in its entirety), bleomycin (Kimura et al., (1972) Cancer 29(1):58-60), gemcitabine (Plunkett et al., (1995) Semin Oncol 22(4 Suppl 11):3-10), platins (e.g., carboplatin, cisplatin, oxaliplatin, satraplatin, picoplatin) (Kelland (2007) Nat Rev Cancer 7(8):573-584), taxanes (e.g., docetaxel, paclitaxel, abraxane) (Abal et al., (2003) Curr Cancer Drug Targets 3(3):193-203) (including albumin-bound versions of taxanes (e.g., albumin-bound paclitaxel), DNA alkylating agents (eg. cyclophosphamide, bendamustine) (Leoni et al., (2008) Clin Cancer Res 14(1):309-317), CHOP (drug combination of cyclophosphamide, doxorubicin hydrochloride, vincristine and prednisone) (Dunleavy (2014) Hematology Am Soc Hematol Educ Program 2014(1):107-112), and fluorouracil and derivatives thereof (Alvarez et al., (2012) Expert Opin Ther Pat 22(2):107-123, which is incorporated herein by reference in its entirety).

In some embodiments, the methods provided herein comprise administering a anti-CD39 antibody at particular doses and dosages disclosed herein and a combination of chemotherapeutic agents. Exemplary combinations of chemotherapeutic agents (or “chemotherapeutic regimens”) include gemcitabine and paclitaxel (including albumin-bound paclitaxel); flouracil and leucovorin (the “FL” chemotherapeutic regimen); leucovorin, fluorouracil and oxaliplatin (the “FOLFOX” chemotherapeutic regimen); leucovorin, fluorouracil, and irinotecan (the “FOLFIRI” chemotherapeutic regimen); leucovorin, fluorouracil, irinotecan and oxaliplatin (the “FOLFIRIFOX” chemotherapeutic regimen); irinotecan, leucovorin, and fluorouracil (the “IFL” chemotherapeutic regimen); and capecitabine and oxaliplatin (the “CAPOX” chemotherapeutic regimen).

In some embodiments, the chemotherapeutic agent is used in combination with the anti-CD39 antibody is gemcitabine and albumin-bound paclitaxel. In some embodiments, the albumin-bound paclitaxel is administered at a dose of 125 mg/m² on days 1, 8, and 15 of a 28-day cycle. In some embodiments, albumin-bound paclitaxel is administered to the subject by intravenous administration. In some embodiments, gemcitabine is administered at a dose of 1000 mg/m² on days 1, 8, and 15 of a 28-day cycle. In some embodiments, gemcitabine is administered to the subject by intravenous administration. In some embodiments, the anti-CD39 antibody, albumin-bound paclitaxel, and gemcitabine are administered sequentially. In some embodiments, the anti-CD39 antibody is administered before albumin-bound paclitaxel and gemcitabine are administered.

In some embodiments, the chemotherapeutic agent induces immunogenic cell death (ICD). In some embodiments, the agent that induces ICD is an anthracycline. In some embodiments, the anthracycline is selected from doxorubicin, daunorubicin, epirubicin, idarubicin, and valrubicin. In some embodiments, the anthracycline is doxorubicin. In some embodiments, the agent that induces ICD is a platinum derivative. In some embodiments, the platinum derivative is selected from oxaliplatin, carboplatin, and cisplatin. In some embodiments, the platinum derivative is oxaliplatin.

Other chemotherapeutic agents suitable for combination and/or co-administration in the disclosed methods include, for example: taxol, cytochalasin B, gramicidin D, ethidium bromide, emetine, mitomycin, etoposide, tenoposide, vincristine, vinblastine, colchicine, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorourac49acarbazineine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide.

In some embodiments the methods provided herein comprise administering a anti-CD39 antibody at particular dosages disclosed herein in conjunction with radiation therapy.

For treatment of cancer, the combinations may be administered in conjunction with one or more additional anti-cancer agents, such as a chemotherapeutic agent, growth inhibitory agent, a tyrosine kinase inhibitor, anti-cancer vaccine such as a gene therapy vaccine, anti-angiogenesis agent and/or anti-neoplastic composition or in conjunction with radiation therapy.

In some embodiments, an anti-inflammatory drug may be administered with the combination, such as a steroid or a non-steroidal anti-inflammatory drug (NSAID). In cases where it is desirable to render aberrantly proliferative cells quiescent in conjunction with or prior to treatment with CD39 antibodies described herein, hormones, antiandrogens and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX®, enzalutamide, apalutamide, abiraterone acetate, bicalutamide, cyproterone acetate, can also be administered to the subject. When employing the methods or compositions described herein, other agents used in the modulation of tumor growth or metastasis in a clinical setting, such as antimimetics, can also be administered as desired.

Such combination therapies noted above encompass combined administration (where two or three or more therapeutic agents are included in the same or separate formulations or compositions), and separate administration, in which case, administration of the anti-CD39 antibody can occur prior to, simultaneously, and/or following, administration of the additional therapeutic agent or agents. In some embodiments, administration of the anti-CD39 antibody and administration of an additional therapeutic agent occur within about one month, or within about one, two or three weeks, or within about one, two, three, four, five, or six days, of each other.

The anti-CD39 antibody (and any additional therapeutic agent) can be administered by any suitable means, including parenteral, intrapulmonary, and intranasal, and, if desired for local treatment, intralesional administration. Parenteral infusions include intramuscular, intravenous, intraarterial, or intraperitonealadministration. Dosages may be administered by any suitable route, e.g. by injections, such as intravenous injections, depending in part on whether the administration is brief or chronic. Various dosages including but not limited to single or multiple administrations over various time-points, bolus administration, and pulse infusion are contemplated herein. In some embodiments of the methods of treating cancer disclosed herein, the anti-CD39 antibody is administered intravenously.

The following additional embodiments are encompassed in their entirety.

Embodiment 1. A method of treating cancer in a human subject in need thereof comprising administering a pharmaceutical composition comprising an anti-CD39 antibody, wherein the antibody is administered at a dose of 20, 70, 200, 700, 1400, or 2000 mg.

Embodiment 2. The method of embodiment 1, wherein the anti-CD39 antibody comprises:
i) HCDR1 comprising the amino acid sequence of SEQ ID NO: 30001;
ii) HCDR2 comprising the amino acid sequence of SEQ ID NO: 30002;
iii) HCDR3 comprising the amino acid sequence of SEQ ID NO: 30003;
iv) LCDR1 comprising the amino acid sequence of SEQ ID NO: 30004;
v) LCDR2 comprising the amino acid sequence of SEQ ID NO: 30005; and
vi) LCDR3 comprising the amino acid sequence of SEQ ID NO: 30006.

Embodiment 3. The method of embodiment 1, wherein the antibody is administered intravenously.

Embodiment 4. The method of embodiment 1 or embodiment 2, wherein the antibody is administered once every 1, 2, 3, 4, 5 or 6 weeks.

Embodiment 5. The method of any one of embodiments 1-3, wherein the antibody is administered once every 2 weeks.

Embodiment 6. The method of any one of the preceding embodiments, wherein the antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 30012 and the VL is at least 90%, 91%, 92%, 93%, 94%, 95%, 96%, 97%, 98%, 99%, or 100% identical to the amino acid sequence of SEQ ID NO: 30018.

Embodiment 7. The method of any one of the preceding embodiments, wherein the VH comprises the amino acid sequence of SEQ ID NO: 30012 and the VL comprises the amino acid sequence of SEQ ID NO: 30018.

Embodiment 8. The method of any one of the preceding embodiments, wherein the antibody is a full-length antibody.

Embodiment 9. The method of any one of the preceding embodiments, wherein the antibody comprises a fully human immunoglobulin G4 (IgG4) antibody.

Embodiment 10. A pharmaceutical composition comprising an anti-CD39 antibody and a pharmaceutically acceptable carrier, wherein the anti-CD39 antibody is formulated for administration at a dose of 20, 70, 200, 700, 1400, or 2000 mg.

Embodiment 11. The pharmaceutical composition of embodiment 10, wherein the anti-CD39 antibody comprises:
i) six CDRs comprising:
   i. HCDR1 comprising the amino acid sequence of SEQ ID NO: 30001;
   ii. HCDR2 comprising the amino acid sequence of SEQ ID NO: 30002;
   iii. HCDR3 comprising the amino acid sequence of SEQ ID NO: 30003;
   iv. LCDR1 comprising the amino acid sequence of SEQ ID NO: 30004;
   v. LCDR2 comprising the amino acid sequence of SEQ ID NO: 30005; and
   vi. LCDR3 comprising the amino acid sequence of SEQ ID NO: 30006; or
ii) a VH comprising the amino acid sequence of SEQ ID NO: 30012 and a VL comprising the amino acid sequence of SEQ ID NO: 30018.

Embodiment 12. The pharmaceutical composition of embodiment 10 or embodiment 11 for use in treating cancer in a human subject in need thereof.

Embodiment 13. The pharmaceutical composition for use of embodiment 12, wherein the anti-CD39 antibody is administered intravenously.

Embodiment 14. The pharmaceutical composition for use of embodiment 12 or embodiment 13, wherein the anti-CD39 antibody is administered once every 1, 2, 3, 4, 5 or 6 weeks.

Embodiment 15. The pharmaceutical composition for use of any one of embodiments 12-14, wherein the anti-CD39 antibody is administered once every two weeks.

Embodiment 16. The method or pharmaceutical composition for use of any one of the preceding embodiments, wherein the cancer is newly diagnosed or non-metastatic.

Embodiment 17. The method or pharmaceutical composition for use of any one of the preceding embodiments, wherein the cancer is advanced.

Embodiment 18. The method or pharmaceutical composition for use of any one of the preceding embodiments, wherein the cancer is refractory.

Embodiment 19. The method or pharmaceutical composition for use of any one of the preceding embodiments, wherein the cancer is metastatic.

Embodiment 20. The method or pharmaceutical composition for use of any one of the preceding embodiments, wherein the cancer is a solid tumor.

Embodiment 21. The method or pharmaceutical composition for use of any one of the preceding embodiments, wherein the cancer is an advanced solid tumor.

Embodiment 22. The method or pharmaceutical composition for use of any one of the preceding embodiments, wherein the cancer is a relapsed solid tumor.

Embodiment 23. The method or pharmaceutical composition for use of any one of the preceding embodiments, wherein the cancer is a refractory solid tumor.

Embodiment 24. The method or pharmaceutical composition for use of any one of the preceding embodiments, wherein the cancer is a metastatic solid tumor.

Embodiment 25. The method or pharmaceutical composition for use of any one of the preceding embodiments, wherein the cancer is carcinoma, lymphoma, blastoma, sarcoma, or leukemia.

Embodiment 26. The method or pharmaceutical composition for use of any one of the preceding embodiments, wherein the cancer is pancreatic cancer.

Embodiment 27. The method or pharmaceutical composition for use of any one of the preceding embodiments, wherein the cancer is gastric cancer.

Embodiment 28. The method or pharmaceutical composition for use of any one of the preceding embodiments, wherein the cancer is prostate cancer.

Embodiment 29. The method or pharmaceutical composition for use of any one of the preceding embodiments, wherein the cancer is endometrial cancer.

Embodiment 30. The method or pharmaceutical composition for use of any one of the preceding embodiments, wherein the cancer is non-small cell lung cancer.

Embodiment 31. The method or pharmaceutical composition for use of any one of the preceding embodiments, wherein the cancer is colorectal cancer.

Embodiment 32. The method or pharmaceutical composition for use of any one of the preceding embodiments, wherein the cancer is ovarian cancer.

Embodiment 33. The method or pharmaceutical composition for use of any one of the preceding embodiments, wherein the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

Embodiment 34. The method or pharmaceutical composition for use of any one of the preceding embodiments, wherein the method or use further comprises administering a second therapy.

Embodiment 35. The method or pharmaceutical composition for use of embodiment 34, wherein the second therapy is a chemotherapeutic agent.

Embodiment 36. The method or pharmaceutical composition for use of embodiment 34 or embodiment 35, wherein the second therapy is gemcitabine.

Embodiment 37. The method or pharmaceutical composition for use of embodiment 34 or embodiment 35, wherein the second therapy is albumin-bound paclitaxel.

Embodiment 38. The method or pharmaceutical composition for use of embodiment 34 or embodiment 35, wherein the second therapy is an antagonist of PD-1 or PD-Li.

Embodiment 39. The method or pharmaceutical composition for use of embodiment 34, wherein the second therapy is an anti-PD-1 antibody.

Embodiment 40. The method or pharmaceutical composition for use of any one of embodiments 1-9 or 12-33, wherein the method or use further comprises administering two additional therapies.

Embodiment 41. The method or pharmaceutical composition for use of embodiment 40, wherein the two additional therapies comprise a chemotherapeutic agent and an antagonist of PD-1 or an antagonist of PD-L1.

Embodiment 42. The method or pharmaceutical composition for use of embodiment 40, wherein the two additional therapies comprise a chemotherapeutic agent and an agent targeting the adenosine axis.

Embodiment 43. The method or pharmaceutical composition for use of embodiment 40, wherein the two additional therapies comprise an antagonist of PD-1 or an antagonist of PD-L1 and an agent targeting the adenosine axis.

Embodiment 44. The method or pharmaceutical composition for use of embodiment 40, wherein one of the two additional therapies comprise an A2AR antagonist, an A2BR antagonist, or a dual A2AR/A2B antagonist.

Embodiment 45. The method or pharmaceutical composition for use of embodiment 40, wherein the two additional therapies comprise at least one chemotherapeutic agent.

Embodiment 46. The method or pharmaceutical composition for use of embodiment 40, wherein the two additional therapies comprise two chemotherapeutic agents.

Embodiment 47. The method or pharmaceutical composition for use of embodiment 45 or embodiment 46, wherein one of the two additional therapies is gemcitabine.

Embodiment 48. The method or pharmaceutical composition for use of embodiment 45 or embodiment 46, wherein one of the two additional therapies is albumin-bound paclitaxel.

Embodiment 49. The method or pharmaceutical composition for use of any one of embodiments 40, or 45-48, wherein the two additional therapies comprise gemcitabine and albumin-bound paclitaxel.

Embodiment 50. The method or pharmaceutical composition for use of any one of embodiments 37, or 48-49, wherein albumin-bound paclitaxel is administered at a dose of 125 mg/m2 on days 1, 8, and 15 of a 28-day cycle.

Embodiment 51. The method or pharmaceutical composition for use of any one of embodiments 37, or 48-50, wherein albumin-bound paclitaxel is administered to the subject by intravenous administration.

Embodiment 52. The method or pharmaceutical composition for use of any one of embodiments 36, 47, or 49, wherein gemcitabine is administered at a dose of 1000 mg/m2 on days 1, 8, and 15 of a 28-day cycle.

Embodiment 53. The method or pharmaceutical composition for use of any one of embodiments 36, 47, 49, or 52, wherein gemcitabine is administered to the subject by intravenous administration.

Embodiment 54. The method or pharmaceutical composition for use of any one of embodiments 49-53, wherein the anti-CD39 antibody, albumin-bound paclitaxel, and gemcitabine are administered sequentially.

Embodiment 55. The method or pharmaceutical composition for use of embodiment 54, wherein the anti-CD39 antibody is administered before albumin-bound paclitaxel and gemcitabine are administered.

Embodiment 56. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 20 mg intravenously every 2 weeks.

Embodiment 57. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 70 mg intravenously every 2 weeks.

Embodiment 58. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 200 mg intravenously every 2 weeks.

Embodiment 59. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 700 mg intravenously every 2 weeks.

Embodiment 60. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 1400 mg intravenously every 2 weeks.

Embodiment 61. A method of treating cancer in a human subject in need thereof comprising administering an anti-CD39 antibody comprising a VH comprising the amino acid sequence of SEQ ID NO: 30012 and a VL comprising the amino acid sequence of SEQ ID NO: 30018, wherein the antibody is administered at a dose of 2000 mg intravenously every 2 weeks.

### III. EXAMPLES

### Example 1. Dose Escalation, Safety, and Tumor Biopsy Expansion Study in Patients with Advanced Solid Tumors

Patients with advanced solid tumors refractory to standard therapy were enrolled in a phase 1 study of clone 22 administered intravenously every 2 weeks (NCT04336098) to establish its preliminary safety as monotherapy and identify a dose and schedule suitable for monotherapy expansion and combination therapy cohorts. Clinical outcomes based on Response Evaluation Criteria in Solid Tumors, and pharmacokinetic (PK) and pharmacodynamic (PD) analyses were evaluated.

### Study Design:

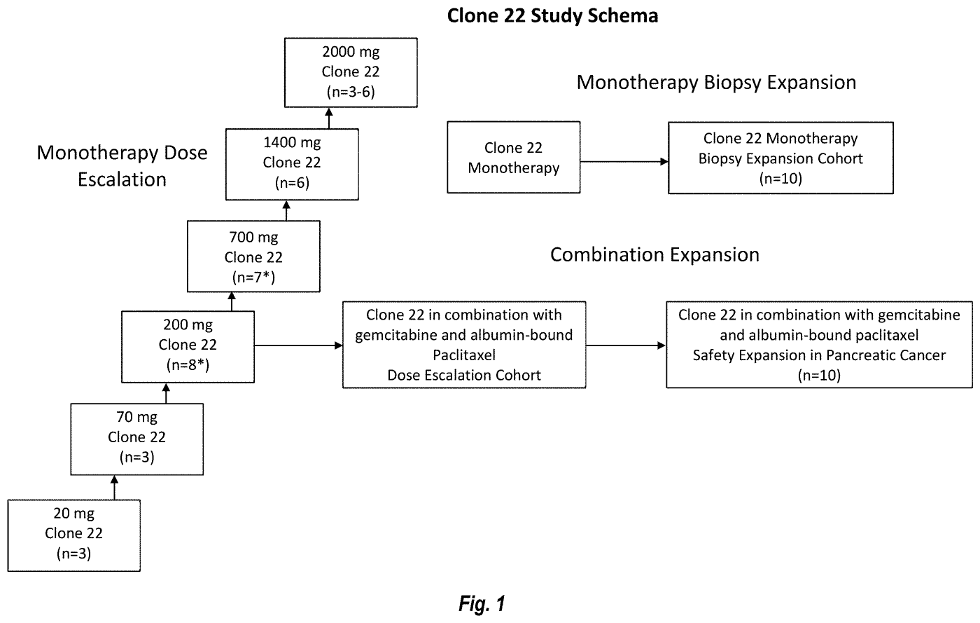

The study was designed as a phase 1 open-label, first-in-human, monotherapy and combination therapy dose escalation, safety, and tumor biopsy expansion study in patients with advanced solid tumors. The study design includes a monotherapy dose escalation portion, a monotherapy tumor biopsy expansion portion, and a combination therapy dose escalation portion. See FIG. 1.

The monotherapy dose escalation portion of the study was designed to evaluate the safety, tolerability, pharmacokinetics (PK), pharmacodynamics, and preliminary efficacy of clone 22 as monotherapy in patients with advanced solid tumors. Patients remain on study drug until disease progression, unacceptable toxicity, or the completion of approximately 24 months of study therapy.

The doses for the monotherapy dose escalation portion of the study are shown in Table 1. The starting dose was 20 mg given intravenously (IV) once every 2 weeks. Under the study design, subsequent clone 22 dose levels may be modified, and additional dose levels and/or schedules may be investigated, based on the recommendation of the Safety Review Committee (SRC) after review of available data.

TABLE 1
Dose Levels

| Dose Level | Clone 22 Dose | | Number of Patients |
|---|---|---|---|
| 1 (starting dose) | 20 | mg | N = 1-6 |
| 2 | 70 | mg | N = 1-6 |
| 3 | 200 | mg | N = 3-6 |
| 4 | 700 | mg | N = 3-6 |
| 5 | 1400 | mg | N = 3-6 |
| 6 | 2000 | mg | N = 3-6 |

Note:
Escalation of dose levels may continue after completion of Dose Level 6 at the recommendation of the Safety Review Committee.

The monotherapy tumor biopsy expansion portion of the study was designed to further evaluate the safety and intratumoral pharmacodynamics of clone 22 monotherapy. Under the design, once a dose level has cleared and enrollment in the next dose level has begun, patients with an advanced solid tumor that is accessible for pretreatment and on-treatment biopsy may be enrolled.

The combination therapy dose escalation portion of the study was designed to evaluate the safety, tolerability, PK, and preliminary efficacy of clone 22 in combination with gemcitabine+albumin-bound paclitaxel in patients with locally advanced or metastatic solid tumors.

Under the design, combination therapy cohorts receive a clone 22 monotherapy dose level that is no higher than the most-recently cleared dose level for clone 22 monotherapy. Gemcitabine+albumin-bound paclitaxel are administered at a dose of albumin-bound paclitaxel 125 mg/m² IV over 30 to 40 minutes followed by gemcitabine 1000 mg/m2 IV on Days 1, 8, and 15 of a 28-day cycle. When scheduled on the same day, gemcitabine and albumin-bound paclitaxel is given after clone 22 has been administered and a 1-hour observation period has elapsed.

The primary study endpoint is dose limiting toxicities (DLTs), including e.g., hematologic toxicities (e.g., decreased neutrophil count, febrile neutropenia, decreased platelet count, and anemia), and nonhematologic toxicities (e.g., nausea, vomiting, diarrhea, fatigue, and immune mediated reactions). Secondary endpoints include adverse event (AEs), antidrug antibodies, electrocardiograms, safety laboratory values, serum concentrations of clone 22, levels of clone 22 target occupancy, objective response rate (ORR), duration of response, disease control rate, progression-free survival (PFS), landmark PFS rate, and levels of intratumoral CD39 enzymatic activity (in patients receiving pretreatment and on-treatment tumor biopsies).

### Interim Monotherapy Results:

As shown in Table 2, twenty-seven (27) patients have been enrolled in the monotherapy arm of the study, and two patients underwent intra-patient dose escalation. The mean age of the patients was 65 years; 10 were male and 17 female; ECOG PS was 0/1 (41%/59%). The median time since initial diagnosis was 38 months and all but two patients were treated with at least one prior systemic therapy; 8 patients were anti-PD-(L)1 experienced.

TABLE 2
Monotherapy Patient Demographics

| Total patients, n | 27 | |
|---|---|---|
| Median age, years (range) | 65 | (20, 80) |
| Sex, n (%) | | |
| Male | 10 | (37%) |
| Female | 17 | (63%) |
| ECOG PS at baseline, n (%) | | |
| 0 | 11 | (41%) |
| 1 | 16 | (59%) |
| Median time since initial diagnosis, months (range) | 38 | (7, 351) |
| Number of prior systemic therapies, n (%) | | |
| 0 | 2 | (7%) |
| 1 | 3 | (11%) |
| 2 | 4 | (15%) |
| 3 | 4 | (15%) |
| ≥4 | 14 | (52%) |
| Prior aPD-1/aPD-L1, n (%) | | |
| Yes | 8 | (30%) |
| No | 19 | (70%) |

As shown in Table 3 and Table 4, clinical outcomes based on Response Evaluation Criteria in Solid Tumors (RECIST v1.1), and pharmacokinetic (PK) and pharmacodynamic (PD) analyses were evaluated. The most common treatment emergent adverse events (“TEAE”), defined as an adverse event that emerges or worsens in the period from the first dose of clone 22 to 30 days after the last dose, across dosing cohorts were low grade fatigue (n=0, 37%), constipation (n=5, 18%) and nausea (n=5, 18%). There were no grade 3 or greater related adverse events, serious adverse events or treatment discontinuations attributed to clone 22. Clone 22 was well-tolerated and no dose-limiting toxicities were observed. Mean time on study was 8 weeks. One patient with non-small cell lung cancer who had previously progressed on chemotherapy and anti-PD-1 therapy remained on study with stable disease for 9 months prior to disease progression.

TABLE 3
Monotherapy Patient Treatment-Emergent
Adverse Event (TEAE) Summary

| Total patients, n | 27 | |
|---|---|---|
| Treatment-emergent adverse event* (TEAE), n (%) | 23 | (85%) |
| Treatment-related AE, n (%) | 14 | (52%) |
| Grade ≥3 TEAE, n (%) | 7 | (26%) |
| Grade ≥3 treatment-related AE, n (%) | 0 | (0%) |
| Serious Treatment- Emergent Adverse Event (TESAE), n | 6 | (22%) |
| (%) | | |
| Treatment-related SAE, n (%) | 0 | (0%) |
| TEAE leading to discontinuation, n (%) | 0 | (0%) |

TABLE 4
Monotherapy Patient Treatment-Emergent Adverse
Events (TEAEs) by Preferred Term

| TEAEs Occurring | | | | |
|---|---|---|---|---|
| in ≥10% of Patients | Grade | Grade | Grade | All |
| by Preferred Term, n (%) | 1 | 2 | 3 | Grades |
| Fatigue | 6 (22%) | 4 (15%) | 0 (0%) | 10 (37%) |
| Constipation | 3 (11%) | 2 (7%) | 0 (0%) | 5 (18%) |
| Nausea | 3 (11%) | 1 (4%) | 1 (4%) | 5 (18%) |
| Vomiting | 2 (7%) | 0 (0%) | 1 (4%) | 3 (11%) |
| Anemia | 0 (0%) | 0 (0%) | 3 (11%) | 3 (11%) |
| Dehydration | 1 (4%) | 2 (7%) | 0 (0%) | 3 (11%) |
| Headache | 2 (7%) | 1 (4%) | 0 (0%) | 3 (11%) |
| Pruritus | 2 (7%) | 1 (4%) | 0 (0%) | 3 (11%) |

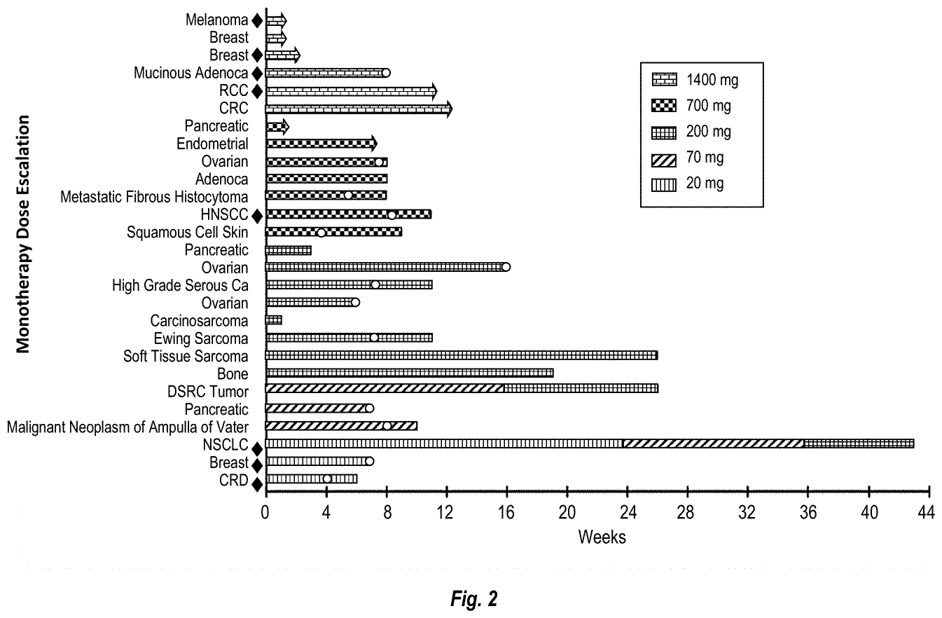

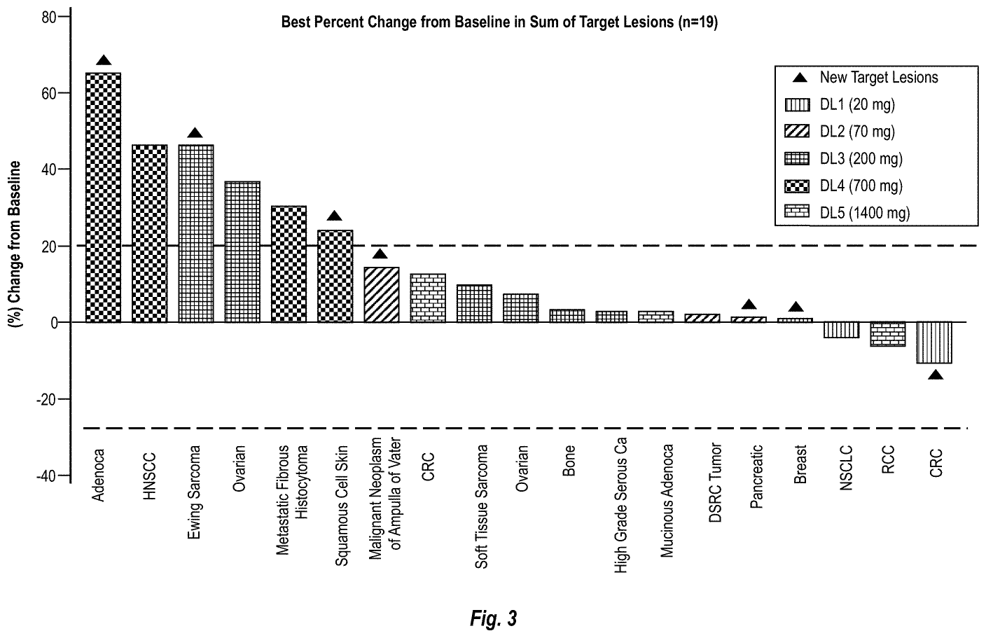

FIG. 2 illustrates individual patients' time and dosing levels on the study. The median time on the study was 8 weeks with a range of 1 to 43 weeks, and 37% of patients had stable disease for longer than 8 weeks. Of the nineteen (19) patients with an evaluable response as of the database snapshot date, 7 patients (35%) had stable disease and 12 patients (60%) had experienced disease progression. FIG. 3 illustrates the best percentage change in target lesions from baseline for the nineteen (19) patients with an evaluable response.

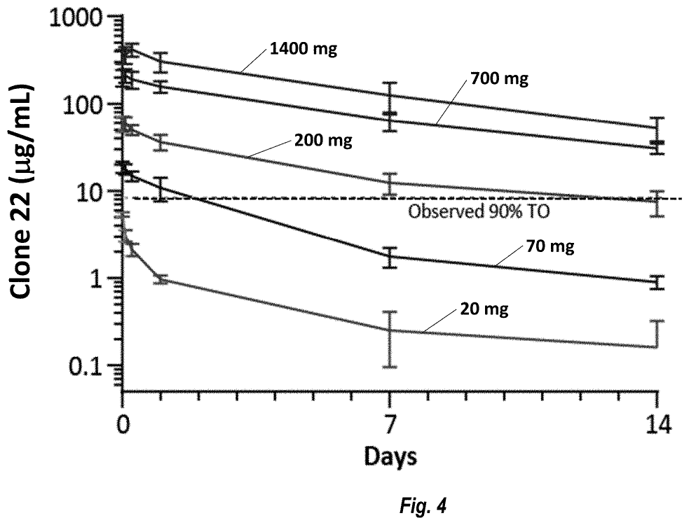

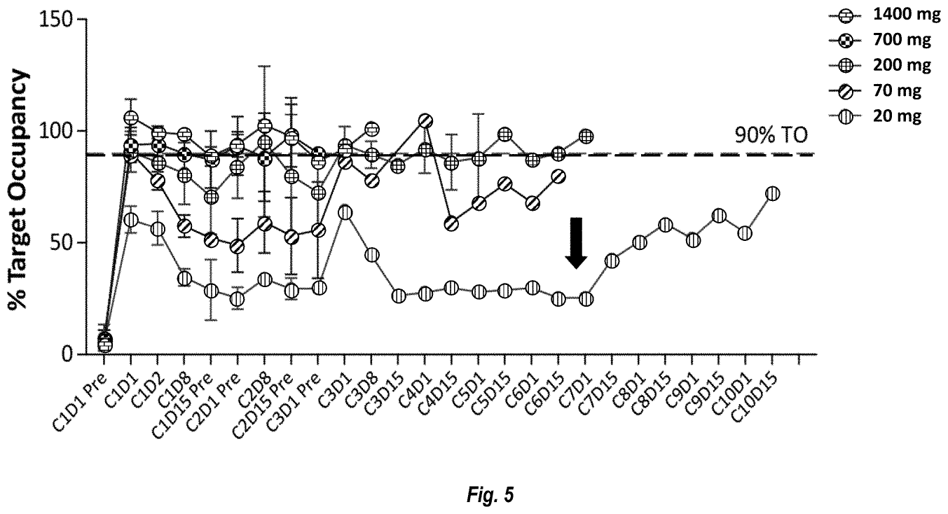

As shown in FIG. 4 and FIG. 5, pharmacokinetics (PK) is linear and correlated strongly with pharmacodynamic (PD) measures of target occupancy. The estimated half-life of clone 22 was 5-7 days. Target occupancy on peripheral blood monocytes (PBMCs) increased in a dose-dependent manner. Maximal target occupancy was observed within 6 hours at doses of 70 mg and above. Target occupancy was maintained near saturation throughout the dosing interval at 200 mg and above.

### Interim Gemcitabine/Albumin-Bound Paclitaxel Combination Results:

Five (5) patients were dosed with clone 22 (200 mg intravenously once every two weeks) in combination with gemcitabine (1000 mg/m² administered as an IV infusion on Days 1, 8, and 15 of a 28-day cycle) and albumin-bound paclitaxel (125 mg/m² administered as an IV infusion over 30 to 40 minutes prior to gemcitabine on days 1, 8, and 15 of a 28-day cycle). Four (4) patients had with follow-up beyond the initial treatment cycle as of the database snapshot date.

Patients treated in the combination arm experienced adverse events as expected, with reported bone marrow suppression and diarrhea.

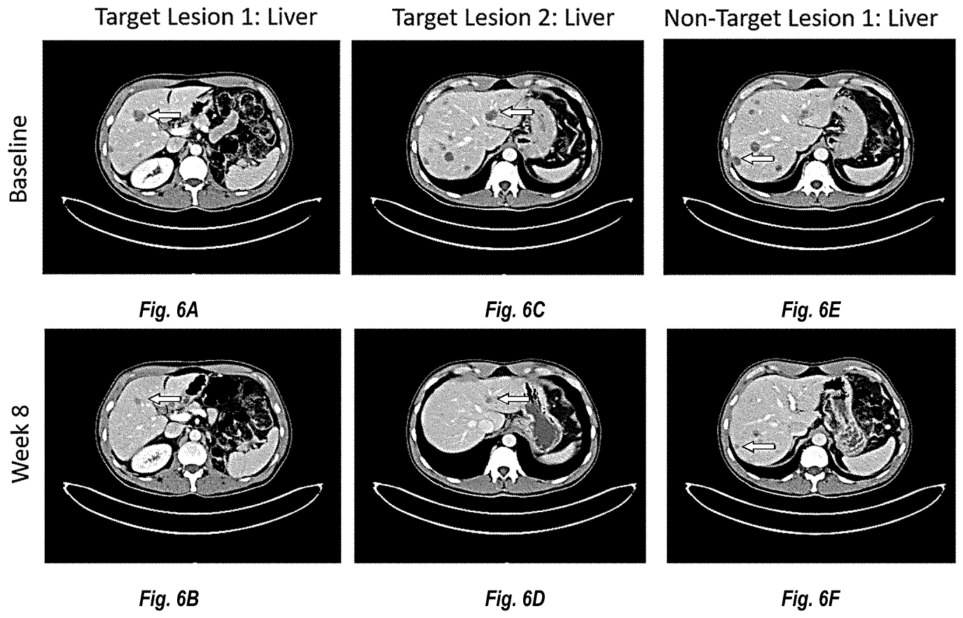

As shown in FIG. 6, a 60-year old male patient with pancreatic cancer with extensive liver metastases whose disease had previously progressed while on FOLFIRINOX chemotherapy regimen had an unconfirmed partial response subsequent to treatment in this combination cohort (clone 22, 200 mg administered as an intravenous injection once every two weeks; gemcitabine, 1000 mg/m² administered as an intravenous [IV] infusion on Days 1, 8, and 15 of a 28-day cycle and albumin-bound paclitaxel, 125 mg/m² administered as an IV infusion over 30 to 40 minutes prior to gemcitabine on days 1, 8, and 15 of a 28-day cycle. The patient experienced approximately 50% tumor shrinkage after two (2) cycles of therapy as reported at the first response evaluation (week 8).

### Updated Interim Monotherapy Results:

Thirty seven (37) patients have been enrolled in the monotherapy arm of the study. Median time on study was 10 weeks. The most common TEAEs in monotherapy were fatigue (35%), nausea (22%), and constipation (19%). No dose-limiting toxicities have been observed, and the monotherapy recommended phase 2 dose was determined to be 1400 mg every 2 weeks (Q2W). PK are linear and correlate strongly with PD measures of target occupancy.

### Additional Updated Interim Monotherapy Results:

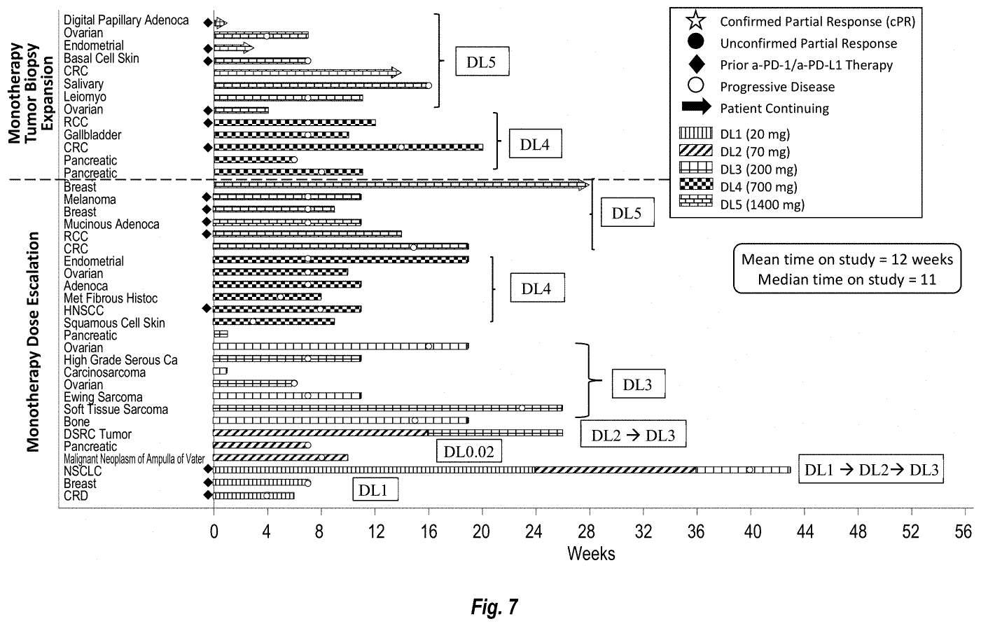

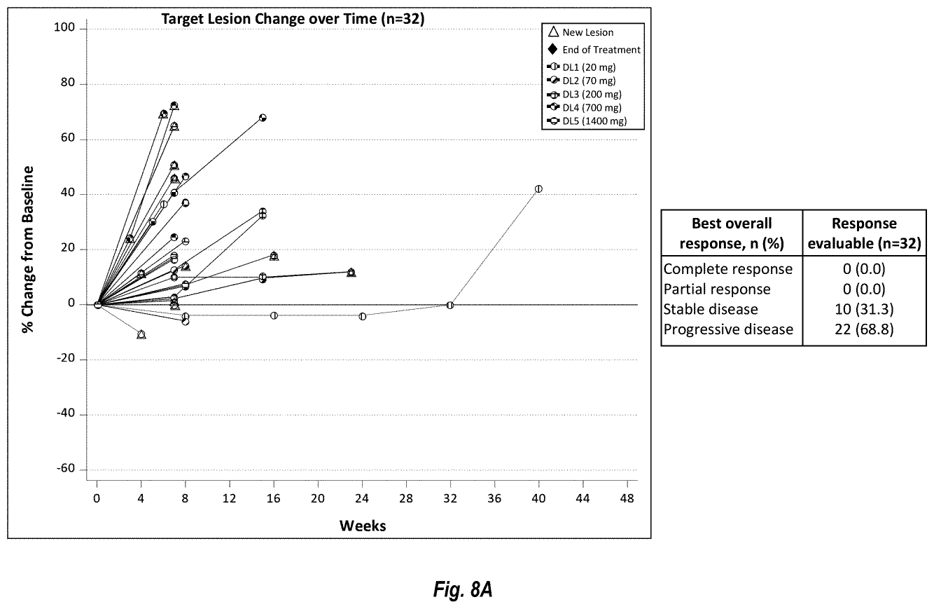

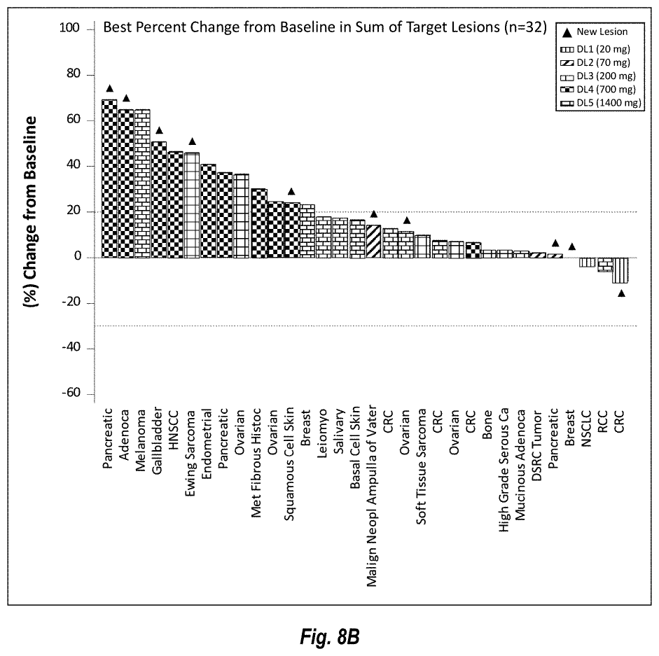

Thirty nine (39) patients have been enrolled in the monotherapy arm of the study. Table 5. Clone 22 monotherapy was well-tolerated at all dose levels tested in patients with advanced solid tumors and had a favorable safety profile. As shown in FIG. 7, thirty nine (39) individuals have been enrolled in the monotherapy arm of the study, and the median time on study was 11 weeks. As shown in FIGS. 8A-8B 10 of 32 evaluable patients (31%) had disease stabilization at eight weeks, with four (12%) persisting beyond 16 weeks, with 1 patient with prolonged disease stabilization beyond 24 weeks with NSCLC whose disease previously progressed on chemotherapy and PD-1 blockade.

TABLE 5
Clone 22 Monotherapy (n = 39)

| | | |
|---|---|---|
| Median age, years (range) | 65 | (20, 80) |
| Sex, n (%) | | |
| Male | 16 | (41%) |
| Female | 23 | (59%) |
| ECOG PS at baseline, n (%) | | |
| 0 | 17 | (45%) |
| 1 | 21 | (55%) |
| Median time since initial diagnosis, months (range) | 42 | (7, 351) |
| Number of prior systemic therapies, n (%) | | |
| 0 | 1 | (2%) |
| 1-3 | 19 | (49%) |
| 4-5 | 9 | (23%) |
| ≥5 | 10 | (26%) |
| Prior aPD-1/aPD-L1, n (%) | | |
| Yes | 14 | (36%) |
| No | 25 | (64%) |

### Updated Interim Gemcitabine/Albumin-Bound Paclitaxel Combination Results:

Nine (9) patients were dosed with clone 22 in combination with gemcitabine (1000 mg/m² administered as an IV infusion on Days 1, 8, and 15 of a 28-day cycle) and albumin-bound paclitaxel (125 mg/m² administered as an IV infusion over 30 to 40 minutes prior to gemcitabine on days 1, 8, and 15 of a 28-day cycle). The most common TEAEs were fatigue (56%), anemia, nausea, and decreased neutrophil and platelet counts (44%, each). No dose-limiting toxicities have been observed. PK are linear and correlate strongly with PD measures of target occupancy.

The previously unconfirmed partial response in the 60-year old male patient with pancreatic cancer with extensive liver metastases whose disease had previously progressed while on FOLFIRINOX chemotherapy regimen was confirmed.

Preliminary data demonstrate that clone 22 is well-tolerated at doses that sustain full target occupancy throughout the dosing interval supporting the initiation of expansion cohorts.

### Updated Interim Gemcitabine/Albumin-Bound Paclitaxel Combination Results:

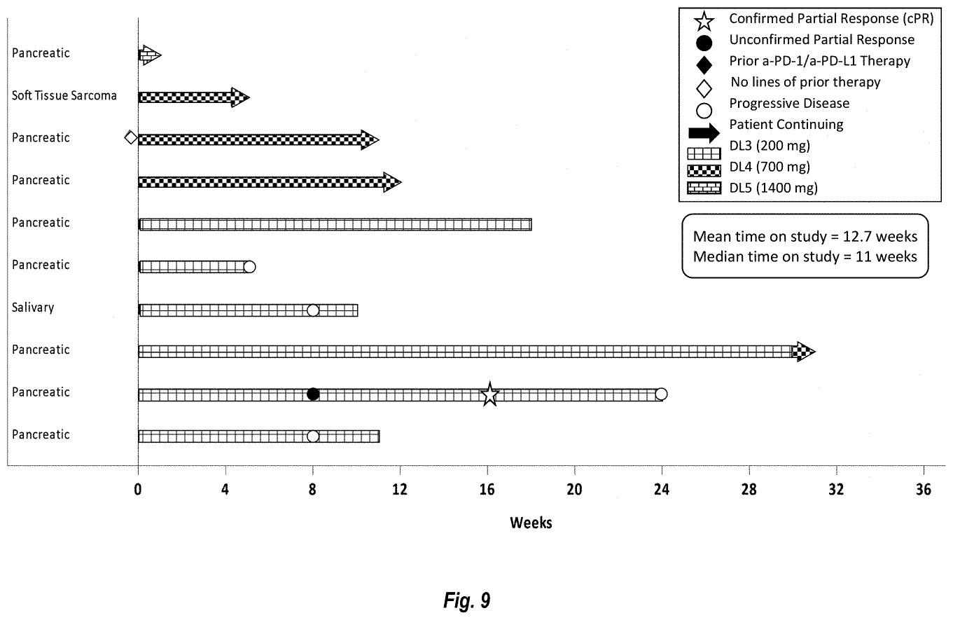

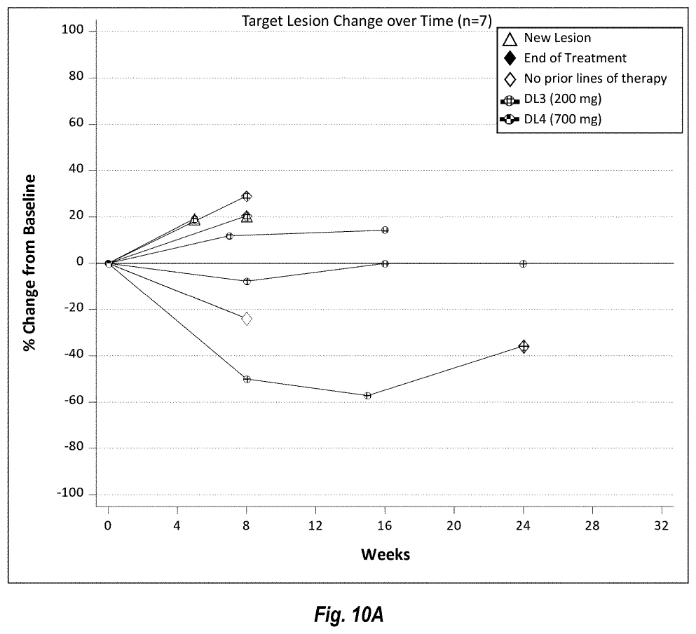

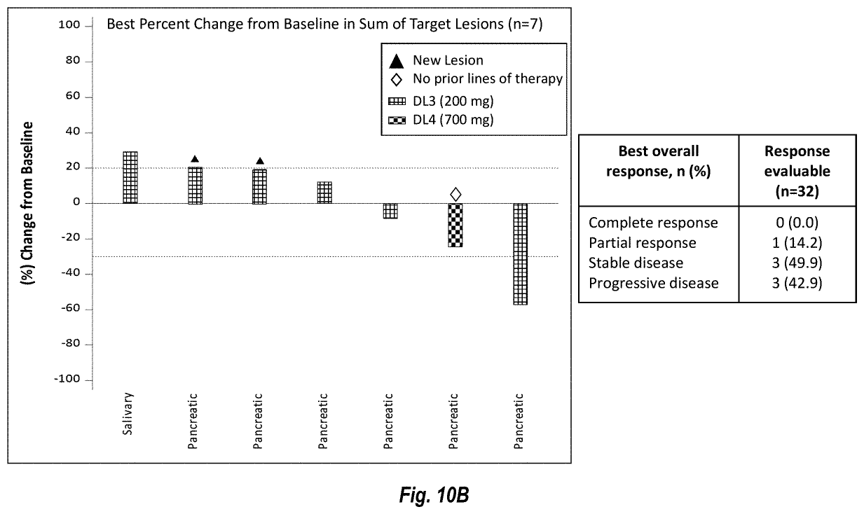

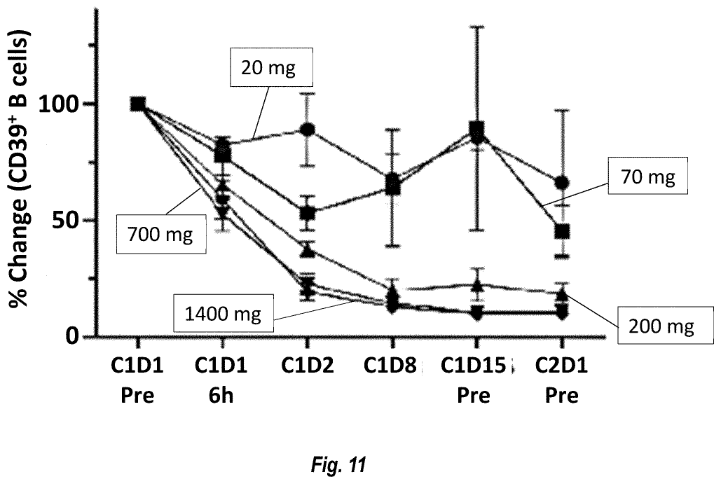

As shown in FIG. 9, ten (10) patients have been enrolled in the clone 22/gemcitabine/albumin-bound paclitaxel triple combination dose escalation study. To date, there has been one confirmed partial response in a patient with pancreateic cancer whose disease progressed on prior chemotherapy (FIG. 9, see star).

There are ongoing expansions enrolling in naïve gastric cancer and in PD-1 relapsed/refractory gastric and NSCLC patients.

Although the foregoing invention has been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

### IV. Table of Sequences

| SEQ | | | |
|---|---|---|---|
| ID | Clone | | |
| NO | No | Description | Sequence |
| 30001 | 22 | VH CDR1 | GTFSSEGIS |
| 30002 | 22 | VH CDR2 | SILPIFGTANYAQKFQG |
| 30003 | 22 | VH CDR3 | AREAGYYRYRYFDL |
| 30004 | 22 | VL CDR1 | RASQSVSSNLA |
| 30005 | 22 | VL CDR2 | GASTRAT |
| 30006 | 22 | VL CDR3 | QQHALWPLT |
| 30007 | 22 | VH FR1 | QVQLVQSGAEVKKPGSSVKVSCKASG |
| 30008 | 22 | VH FR2 | WVRQAPGQGLEWMG |
| 30009 | 22 | VH FR3 | RVTITADESTSTAYMELSSLRSEDTAVYYC |
| 30010 | 22 | VH FR4 | WGRGTLVTVSS |
| 30011 | 22 | VH DNA | CAAGTGCAGTTGGTGCAGTCCGGAGCCGAAGTCAAGAAGCCC |
| | | | GGGTCGAGCGTGAAAGTGTCCTGCAAGGCTTCTGGTGGAACCT |
| | | | TCTCAAGCGAAGGGATCAGCTGGGTCAGACAGGCGCCGGGCC |
| | | | AGGGTCTGGAGTGGATGGGTTCCATTCTCCCGATCTTCGGAAC |
| | | | CGCCAATTACGCCCAGAAGTTCCAGGGTCGCGTGACCATCACC |
| | | | GCCGACGAAAGCACCTCGACGGCCTATATGGAATTGTCGTCCC |
| | | | TGCGGTCGGAAGATACAGCGGTGTACTACTGTGCGCGGGAAGC |
| | | | CGGGTACTACCGCTACCGCTACTTCGATCTGTGGGGAAGGGGA |
| | | | ACTCTCGTGACTGTGTCGAGCG |
| 30012 | 22 | VH Protein | QVQLVQSGAEVKKPGSSVKVSCKASGGTFSSEGISWVRQAPGQG |
| | | | LEWMGSILPIFGTANYAQKFQGRVTITADESTSTAYMELSSLRSED |
| | | | TAVYYCAREAGYYRYRYFDLWGRGTLVTVSS |
| 30013 | 22 | VL FR1 | EIVMTQSPATLSVSPGERATLSC |
| 30014 | 22 | VL FR2 | WYQQKPGQAPRLLIY |
| 30015 | 22 | VL FR3 | GIPARFSGSGSGTEFTLTISSLQSEDFAVYYC |
| 30016 | 22 | VL FR4 | FGGGTKVEIK |
| 30017 | 22 | VL DNA | GAAATAGTGATGACGCAGTCTCCAGCCACCCTGTCTGTGTCTC |
| | | | CAGGGGAAAGAGCCACCCTCTCCTGCAGGGCCAGTCAGAGTGT |
| | | | TAGCAGCAACTTAGCCTGGTACCAGCAGAAACCTGGCCAGGCT |
| | | | CCCAGGCTCCTCATCTATGGTGCATCCACCAGGGCCACTGGTA |
| | | | TCCCAGCCAGGTTCAGTGGCAGTGGGTCTGGGACAGAGTTCAC |
| | | | TCTCACCATCAGCAGCCTGCAGTCTGAAGATTTTGCAGTTTATT |
| | | | ACTGTCAGCAGCACGCCCTCTGGCCTCTCACTTTTGGCGGAGG |
| | | | GACCAAGGTTGAGATCAAA |
| 30018 | 22 | VL Protein | EIVMTQSPATLSVSPGERATLSCRASQSVSSNLAWYQQKPGQAPR |
| | | | LLIYGASTRATGIPARFSGSGSGTEFTLTISSLQSEDFAVYYCQQHA |
| | | | LWPLTFGGGTKVEIK |
| 30019 | 22 | Heavy | QVQLVQSGAEVKKPGSSVKVSCKASGGTFSSEGISWVRQAPGQG |
| | | Chain | LEWMGSILPIFGTANYAQKFQGRVTITADESTSTAYMELSSLRSED |
| | | Protein | TAVYYCAREAGYYRYRYFDLWGRGTLVTVSSASTKGPSVFPLAP |
| | | | CSRSTSESTAALGCLVKDYFPEPVTVSWNSGALTSGVHTFPAVLQ |
| | | | SSGLYSLSSVVTVPSSSLGTKTYTCNVDHKPSNTKVDKRVESKYG |
| | | | PPCPPCPAPEFLGGPSVFLFPPKPKDTLMISRTPEVTCVVVDVSQE |
| | | | DPEVQFNWYVDGVEVHNAKTKPREEQFNSTYRVVSVLTVLHQD |
| | | | WLNGKEYKCKVSNKGLPSSIEKTISKAKGQPREPQVYTLPPSQEE |
| | | | MTKNQVSLTCLVKGFYPSDIAVEWESNGQPENNYKTTPPVLDSD |
| | | | GSFFLYSRLTVDKSRWQEGNVFSCSVMHEALHNHYTQKSLSLSL |
| | | | G |
| 30020 | 22 | Heavy | CAAGTGCAGTTGGTGCAGTCCGGAGCCGAAGTCAAGAAGCCC |
| | | Chain DNA | GGGTCGAGCGTGAAAGTGTCCTGCAAGGCTTCTGGTGGAACCT |
| | | | TCTCAAGCGAAGGGATCAGCTGGGTCAGACAGGCGCCGGGCC |
| | | | AGGGTCTGGAGTGGATGGGTTCCATTCTCCCGATCTTCGGAAC |
| | | | CGCCAATTACGCCCAGAAGTTCCAGGGTCGCGTGACCATCACC |
| | | | GCCGACGAAAGCACCTCGACGGCCTATATGGAATTGTCGTCCC |
| | | | TGCGGTCGGAAGATACAGCGGTGTACTACTGTGCGCGGGAAGC |
| | | | CGGGTACTACCGCTACCGCTACTTCGATCTGTGGGGAAGGGGA |
| | | | ACTCTCGTGACTGTGTCGAGCGCCAGCACCAAGGGACCCAGCG |
| | | | TGTTCCCGCTGGCCCCTTGTTCACGATCCACTTCCGAAAGCACC |
| | | | GCTGCCCTTGGCTGCCTTGTCAAGGACTACTTCCCTGAGCCCGT |
| | | | CACTGTGTCGTGGAACAGCGGAGCTCTGACCTCCGGCGTCCAC |
| | | | ACCTTCCCGGCTGTGCTCCAGTCCTCCGGCCTGTACTCACTGTC |
| | | | CTCGGTGGTCACCGTGCCCTCCTCCTCCCTCGGTACCAAGACTT |
| | | | ATACCTGCAACGTGGACCACAAGCCCTCCAACACCAAAGTGGA |
| | | | TAAGAGAGTGGAGAGCAAATACGGACCTCCCTGCCCTCCTTGC |
| | | | CCTGCGCCTGAGTTTCTGGGCGGACCATCCGTCTTTCTGTTCCC |
| | | | ACCGAAGCCCAAGGACACCCTCATGATCTCCCGGACCCCCGAA |
| | | | GTGACCTGTGTGGTGGTGGACGTGTCACAGGAGGACCCTGAAG |
| | | | TGCAGTTTAATTGGTACGTCGACGGCGTGGAAGTGCATAACGC |
| | | | AAAGACCAAGCCGCGGGAGGAACAGTTCAACTCAACCTACCG |
| | | | CGTGGTGTCCGTGCTGACTGTGCTGCACCAGGACTGGCTGAAC |
| | | | GGAAAGGAGTATAAGTGCAAAGTCTCCAACAAGGGACTGCCG |
| | | | AGCAGCATCGAGAAAACCATTTCAAAAGCCAAGGGCCAGCCG |
| | | | AGAGAGCCCCAAGTGTACACTCTGCCGCCGAGCCAAGAGGAA |
| | | | ATGACCAAGAACCAAGTGTCCCTCACTTGCCTGGTCAAGGGCT |
| | | | TCTACCCGTCGGACATCGCCGTGGAGTGGGAAAGCAACGGCCA |
| | | | GCCGGAAAACAACTACAAGACTACCCCTCCCGTCCTCGACTCC |
| | | | GACGGGTCCTTCTTCCTCTACTCCCGGCTGACTGTGGATAAGTC |
| | | | ACGGTGGCAGGAGGGAAACGTGTTCTCGTGCTCCGTGATGCAC |
| | | | GAAGCCCTGCACAACCACTACACGCAGAAGTCCCTGTCCTTGT |
| | | | CCCTGGGG |
| 30021 | 22 | Light Chain | EIVMTQSPATLSVSPGERATLSCRASQSVSSNLAWYQQKPGQAPR |
| | | Protein | LLIYGASTRATGIPARFSGSGSGTEFTLTISSLQSEDFAVYYCQQHA |
| | | | LWPLTFGGGTKVEIKRTVAAPSVFIFPPSDEQLKSGTASVVCLLNN |
| | | | FYPREAKVQWKVDNALQSGNSQESVTEQDSKDSTYSLSSTLTLSK |
| | | | ADYEKHKVYACEVTHQGLSSPVTKSFNRGEC |
| 30022 | 22 | Light Chain | GAAATAGTGATGACGCAGTCTCCAGCCACCCTGTCTGTGTCTC |
| | | DNA | CAGGGGAAAGAGCCACCCTCTCCTGCAGGGCCAGTCAGAGTGT |
| | | | TAGCAGCAACTTAGCCTGGTACCAGCAGAAACCTGGCCAGGCT |
| | | | CCCAGGCTCCTCATCTATGGTGCATCCACCAGGGCCACTGGTA |
| | | | TCCCAGCCAGGTTCAGTGGCAGTGGGTCTGGGACAGAGTTCAC |
| | | | TCTCACCATCAGCAGCCTGCAGTCTGAAGATTTTGCAGTTTATT |
| | | | ACTGTCAGCAGCACGCCCTCTGGCCTCTCACTTTTGGCGGAGG |
| | | | GACCAAGGTTGAGATCAAACGTACGGTGGCCGCTCCCTCCGTG |
| | | | TTCATCTTCCCACCCTCCGACGAGCAGCTGAAGTCCGGCACCG |
| | | | CCTCCGTCGTGTGCCTGCTGAACAACTTCTACCCTCGCGAGGCC |
| | | | AAAGTGCAGTGGAAAGTGGACAACGCCCTGCAGTCCGGCAAC |
| | | | TCCCAGGAATCCGTCACCGAGCAGGACTCCAAGGACAGCACCT |
| | | | ACTCCCTGTCCTCCACCCTGACCCTGTCCAAGGCCGACTACGA |
| | | | GAAGCACAAAGTGTACGCCTGCGAAGTGACCCACCAGGGCCT |
| | | | GTCCAGCCCCGTGACCAAGTCCTTCAACCGGGGCGAGTGC |
| 30023 | 22 | Heavy | GATATCGCCACCATGGCCTCTCCAGCTCAGCTGCTGTTTC |
| | | (5’ and 3’ | TGCTGCTGCTGTGGCTGCCTGACGGCGTGCACGCACAAGTGCA |
| | | EcoRV | GTTGGTGCAGTCCGGAGCCGAAGTCAAGAAGCCCGGGTCGAG |
| | | restriction | CGTGAAAGTGTCCTGCAAGGCTTCTGGTGGAACCTTCTCAAGC |
| | | sites (bold), | GAAGGGATCAGCTGGGTCAGACAGGCGCCGGGCCAGGGTCTG |
| | | Kozak | GAGTGGATGGGTTCCATTCTCCCGATCTTCGGAACCGCCAATT |
| | | sequence | ACGCCCAGAAGTTCCAGGGTCGCGTGACCATCACCGCCGACGA |
| | | (italics); | AAGCACCTCGACGGCCTATATGGAATTGTCGTCCCTGCGGTCG |
| | | signal | GAAGATACAGCGGTGTACTACTGTGCGCGGGAAGCCGGGTACT |
| | | sequence | ACCGCTACCGCTACTTCGATCTGTGGGGAAGGGGAACTCTCGT |
| | | (underline)) | GACTGTGTCGAGCGCCAGCACCAAGGGACCCAGCGTGTTCCCG |
| | | Chain DNA | CTGGCCCCTTGTTCACGATCCACTTCCGAAAGCACCGCTGCCCT |
| | | | TGGCTGCCTTGTCAAGGACTACTTCCCTGAGCCCGTCACTGTGT |
| | | | CGTGGAACAGCGGAGCTCTGACCTCCGGCGTCCACACCTTCCC |
| | | | GGCTGTGCTCCAGTCCTCCGGCCTGTACTCACTGTCCTCGGTGG |
| | | | TCACCGTGCCCTCCTCCTCCCTCGGTACCAAGACTTATACCTGC |
| | | | AACGTGGACCACAAGCCCTCCAACACCAAAGTGGATAAGAGA |
| | | | GTGGAGAGCAAATACGGACCTCCCTGCCCTCCTTGCCCTGCGC |
| | | | CTGAGTTTCTGGGCGGACCATCCGTCTTTCTGTTCCCACCGAAG |
| | | | CCCAAGGACACCCTCATGATCTCCCGGACCCCCGAAGTGACCT |
| | | | GTGTGGTGGTGGACGTGTCACAGGAGGACCCTGAAGTGCAGTT |
| | | | TAATTGGTACGTCGACGGCGTGGAAGTGCATAACGCAAAGACC |
| | | | AAGCCGCGGGAGGAACAGTTCAACTCAACCTACCGCGTGGTGT |
| | | | CCGTGCTGACTGTGCTGCACCAGGACTGGCTGAACGGAAAGGA |
| | | | GTATAAGTGCAAAGTCTCCAACAAGGGACTGCCGAGCAGCATC |
| | | | GAGAAAACCATTTCAAAAGCCAAGGGCCAGCCGAGAGAGCCC |
| | | | CAAGTGTACACTCTGCCGCCGAGCCAAGAGGAAATGACCAAG |
| | | | AACCAAGTGTCCCTCACTTGCCTGGTCAAGGGCTTCTACCCGTC |
| | | | GGACATCGCCGTGGAGTGGGAAAGCAACGGCCAGCCGGAAAA |
| | | | CAACTACAAGACTACCCCTCCCGTCCTCGACTCCGACGGGTCC |
| | | | TTCTTCCTCTACTCCCGGCTGACTGTGGATAAGTCACGGTGGCA |
| | | | GGAGGGAAACGTGTTCTCGTGCTCCGTGATGCACGAAGCCCTG |
| | | | CACAACCACTACACGCAGAAGTCCCTGTCCTTGTCCCTGGGGA |
| | | | AGTAATGAGATATC |

1.-68. (canceled)

69. A method of treating cancer in a human subject in need thereof comprising administering a pharmaceutical composition comprising an anti-CD39 antibody or fragment thereof, wherein the antibody is administered at a dose of 1400 mg or 2000 mg.

70. The method of claim 69, wherein the anti-CD39 antibody or fragment thereof comprises:
(a) HCDR1 comprising the amino acid sequence of SEQ ID NO: 30001;
(b) HCDR2 comprising the amino acid sequence of SEQ ID NO: 30002;
(c) HCDR3 comprising the amino acid sequence of SEQ ID NO: 30003;
(d) LCDR1 comprising the amino acid sequence of SEQ ID NO: 30004;
(e) LCDR2 comprising the amino acid sequence of SEQ ID NO: 30005; and
(f) LCDR3 comprising the amino acid sequence of SEQ ID NO: 30006.

71. The method of claim 69, wherein the antibody or fragment thereof is administered intravenously.

72. The method of claim 69, wherein the antibody or fragment thereof is administered once every 1, 2, 3, 4, 5 or 6 weeks.

73. The method of claim 69, wherein the antibody or fragment thereof comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 30012 and the VL is at least 90%, 91%, 92%, 93%, 94%, 95%, 96%, 97%, 98%, 99%, or 100% identical to the amino acid sequence of SEQ ID NO: 30018.

74. The method of claim 69, wherein the antibody or fragment thereof comprises a fully human immunoglobulin G4 (IgG4) antibody.

75. A pharmaceutical composition comprising an anti-CD39 antibody or fragment thereof and a pharmaceutically acceptable carrier, wherein the anti-CD39 antibody or fragment thereof is formulated for administration at a dose of 1400 mg or 2000 mg.

76. The pharmaceutical composition of claim 75, wherein the anti-CD39 antibody or fragment thereof comprises:
(a) six CDRs comprising:
i. HCDR1 comprising the amino acid sequence of SEQ ID NO: 30001;
ii. HCDR2 comprising the amino acid sequence of SEQ ID NO: 30002;
iii. HCDR3 comprising the amino acid sequence of SEQ ID NO: 30003;
iv. LCDR1 comprising the amino acid sequence of SEQ ID NO: 30004;
v. LCDR2 comprising the amino acid sequence of SEQ ID NO: 30005; and
vi. LCDR3 comprising the amino acid sequence of SEQ ID NO: 30006; or
(b) a VH comprising the amino acid sequence of SEQ ID NO: 30012 and a VL comprising the amino acid sequence of SEQ ID NO: 30018.

77. The method of claim 69, wherein the cancer is:
(a) newly diagnosed or non-metastatic;
(b) advanced;
(c) refractory;
(d) metastatic;
(e) a solid tumor;
(f) an advanced solid tumor;
(g) a relapsed solid tumor;
(h) a refractory solid tumor;
(i) a metastatic solid tumor;
(j) carcinoma, lymphoma, blastoma, sarcoma, or leukemia;
(k) pancreatic cancer;
(l) gastric cancer;
(m) prostate cancer;
(n) endometrial cancer;
(o) non-small cell lung cancer;
(p) colorectal cancer;
(q) ovarian cancer; and/or
(r) squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

78. The method of claim 69, wherein the method or use further comprises administering a second therapy.

79. The method of claim 78, wherein the second therapy is:
(a) a chemotherapeutic agent;
(b) gemcitabine;
(c) albumin-bound paclitaxel;
(d) an antagonist of PD-1 or PD-Li; or
(e) an anti-PD-1 or anti-PD-L1 antibody.

80. The method of claim 69, wherein the method or use further comprises administering two additional therapies.

81. The method of claim 80, wherein the two additional therapies comprise:
(a) a chemotherapeutic agent and an antagonist of PD-1 or an antagonist of PD-L1;
(b) a chemotherapeutic agent and an agent targeting the adenosine axis;
(c) an antagonist of PD-1 or an antagonist of PD-L1 and an agent targeting the adenosine axis;
(d) at least one chemotherapeutic agent; or
(e) two chemotherapeutic agents.

82. The method or of claim 80, wherein one of the two additional therapies comprise:
(a) an A2AR antagonist, an A2BR antagonist, or a dual A2AR/A2B antagonist;
(b) gemcitabine; or
(c) albumin-bound paclitaxel.

83. The method of claim 80, wherein the two additional therapies comprise gemcitabine and albumin-bound paclitaxel.

84. The method of claim 78, wherein albumin-bound paclitaxel is administered at a dose of 125 mg/m² on days 1, 8, and 15 of a 28-day cycle.

85. The method of claim 78, wherein gemcitabine is administered at a dose of 1000 mg/m² on days 1, 8, and 15 of a 28-day cycle.

86. The method of claim 78, wherein albumin-bound paclitaxel or gemcitabine is administered to the subject by intravenous administration.

87. The method of claim 83, wherein the anti-CD39 antibody, albumin-bound paclitaxel, and gemcitabine are administered sequentially.

88. The method of claim 83, wherein the anti-CD39 antibody is administered before albumin-bound paclitaxel and gemcitabine are administered.

89. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 1400 mg intravenously every 2 weeks.

90. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 2000 mg intravenously every 2 weeks.

91. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 1400 mg intravenously every 2 weeks, and further administering one or two chemotherapeutic agents.

92. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 2000 mg intravenously every 2 weeks, and further administering one or two chemotherapeutic agents.

93. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 1400 mg intravenously every 2 weeks, and further administering gemcitabine and albumin-bound paclitaxel.

94. The method of claim 69, wherein the anti-CD39 antibody comprises a VH comprising the amino acid sequence of SEQ ID NO: 30012 and a VL comprising the amino acid sequence of SEQ ID NO: 30018, wherein the antibody is administered at a dose of 2000 mg intravenously every 2 weeks, and further administering gemcitabine and albumin-bound paclitaxel.
